SEQUENCE LISTING (10 sequences)

SEQ ID NO: 1 - DNA, 20 bp, Artificial Sequence
  PCR primer, STC-1 forward
cagctccccaatcacttctc

SEQ ID NO: 2 - DNA, 21 bp, Artificial Sequence
  PCR primer, STC-1 reverse
tctccatcaggctgtctctga

SEQ ID NO: 3 - DNA, 23 bp, Artificial Sequence
  PCR primer, GAPDH forward
tcaacggatttggtcgtattggg

SEQ ID NO: 4 - DNA, 20 bp, Artificial Sequence
  PCR primer, GADPH reverse
tgattttggagggatctcgc

SEQ ID NO: 5 - DNA, 21 bp, Artificial Sequence
  RNA probe with deoxynucleotide label
gggaaaagcauucgucaaatt

SEQ ID NO: 6 - DNA, 21 bp, Artificial Sequence
  RNA probe with deoxynucleotide label
uuugacgaaugcuuuuccctg

SEQ ID NO: 7 - DNA, 21 bp, Artificial Sequence
  RNA probe with deoxynucleotide label
ggucuaacuguggaauauatt

SEQ ID NO: 8 - DNA, 21 bp, Artificial Sequence
  RNA probe with deoxynucleotide label
uauauuccacaguuagacctt

SEQ ID NO: 9 - DNA, 21 bp, Artificial Sequence
  RNA probe with deoxynucleotide label
cgacuaaccuaucuaugaatt

SEQ ID NO: 10 - DNA, 21 bp, Artificial Sequence
  RNA probe with deoxynucleotide label
uucauagauagguuagucgtt

(19) United States
(12) Patent Application Publication
(10) Pub. No.: US 2011/0223138 A1
(43) Pub. Date: Sep. 15, 2011

(54) Mesenchymal stem cells that express increased amounts of anti-apoptotic proteins

(76) Inventors: Darwin J. Prockop, Philadelphia, PA (US); Gregory J. Block, Seattle, WA (US); Shinya Ohkouchi, Sendai (JP); Hosoon Choi, Belton, TX (US)

(21) Appl. No.: 12/928,740

(22) Filed: Dec. 17, 2010

Related U.S. Application Data
(60) Provisional application No. 61/287,507, filed on Dec. 17, 2009.

(51) Int. Cl. A61K 35/12; C12N 5/0775; A61K 38/02; A61P 35/00; A61P 43/00; A61P 29/00; A61P 9/00

(52) U.S. Cl. 424/93.7; 435/325; 514/19.3; 514/19.5; 514/19.4; 514/1.1

(57) ABSTRACT

The invention includes a purified population of mesenchymal stromal cells that have been cultured under conditions, such as having been contacted with an apoptotic cell, to express increased levels of at least one anti-apoptotic protein. Such mesenchymal stem cells may be used to treat diseases, disorders, or conditions associated with apoptosis or with unresolved inflammation.

This application claims priority based on provisional application Ser. No. 61/287,507, filed Dec. 17, 2009, the contents of which are incorporated by reference in their entirety.

### STATEMENT REGARDING FEDERALLY SPONSORED RESEARCH OR DEVELOPMENT

This invention was made, in part, using fluids obtained from the U.S. Government (National Institutes of Health Grant Nos. HL073755, HL073252, and P01-HL075161), and the U.S. Government therefore has certain rights in this invention.

### BACKGROUND OF THE INVENTION

Multipotent adherent cells from the bone marrow, known as mesenchymal stem cells or multipotent stromal cells (MSCs), are easily expanded ex vivo and maintain their ability to differentiate into a variety of cell phenotypes [Owen et. al., 1988, Ciba Found Symp; 136:42-60; Owen, 1988, J Cell Sci 10:63-76]. Initial observations suggested that MSCs might repair injured tissues through mechanisms involving differentiation and perhaps fusion [Spees et. al., 2003, Proc Natl Acad Sci USA 100:2397-2402]. Subsequent observations, however, demonstrated that the cells produced functional improvement in several disease models without much evidence of long-term engraftment [Chopp et. al., 2000, Neuroreport 11:3001-3005; Horwitz et. al., 2002, Proc Natl Acad Sci USA 99:8932-8937; Iso et. al., 2007, Biochem. Biophys, Res. Commun. 354:700-706]. The results suggest that MSCs repaired tissues by multiple interactions that included secretion of paracrine factors to enhance regeneration of injured cells, to stimulate the proliferation and differentiation of the stem-like progenitor cells found in most tissues [Munoz et. al., 2005, Proc Natl Acad Sci USA 102:18171-18176; Lee, et. al., 2006, Blood 107:2153-2161], to decrease immune reactions [Le Blanc, 2006, Cytotherapy 8:559-561], and to decrease inflammatory reactions [Ortiz et. al., 2007, Proc Natl Acad Sci USA 104:11002-11007; Gupta et. al., 2007 J Immunol 179:1855-1863; Xu et. al., 2007, Cell Res 17:240-248]. Reports that MSCs decreased apoptosis were of special interest. For example, MSCs that were engineered to overexpress AKT decreased apoptosis in a mouse model of myocardial infarction [Noiseux et. al., 2006, Mol Ther 14:840-850] by secreting the secreted frizzled-related protein-2, an antagonist of Wnt signaling [Mirotsou et. al., 2007, Proc Natl Acad Sci USA 104:1643-1648]. Also, conditioned medium from human MSCs was shown to contain paracrine factors that inhibited apoptosis in hypoxic human aortic endothelial cells that were not fully defined [Hung et. al., 2007, Stem Cells 25:2363-2370].

The present invention provides the necessary data to establish that MSCs have added therapeutic benefit in treating diseases.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiment(s) which are presently preferred. It should be understood, however, that the invention is not limited to the precise arrangements and instrumentalities shown.

FIG. 1. MSCs reduce apoptosis of UV-Fib. (A): Fibroblasts were incubated on a transwell filter, UV-irradiated, and then transferred for coculture with MSCs in a six-well dish. Forty-eight hours later, viability and apoptosis were assayed after labeling for annexin V staining and propidium iodide (PI) incorporation by flow cytometry (B): Quantification of annexin V/PI-positive cells ₊p<05 Error bars=SD. (C): Irradiated fibroblasts were incubated alone or in transwell cocultures with Fib. (D): Representative phase-contrast images of UV-Fib incubated alone and in coculture with MSCs or Fib. Scale bar=100 μm. Magnification ×40. (E): Microarray heat map analysis of shared genes from two MSC donors upregulated or downregulated more than 2.0-fold when incubated either alone (lanes 1, 4), in cocultures with Fib (lanes 2, 5), or in cocultures with irradiated fibroblasts (lanes 3, 6); n=1 (F): Venn diagram of genes upregulated 2.0-fold in each MSC donor cell line when cocultured with UV-Fib versus Fib. Genes upregulated in MSCs from donor 1 (green), donor 2 (red), and both donors (yellow); n=1 Data are from experiments performed with MSC donor 1, unless otherwise stated. Abbreviations: Fib, naïve fibroblasts; MSC, multipotent stromal cells in transwell coculture, MSC CdM, conditioned medium from naïve multipotent stromal cells; UV-Fib, irradiated fibroblasts.

FIG. 2. Upregulation and secretion of STC-1 by MSCs is required but not sufficient for reduction of apoptosis of UV-Fib. (A): Western blot analyses of cell lysates. Left panel: Fibroblasts incubated alone or with UV-Fib, Middle panel: MSCs incubated alone, with Fib or with UV-Fib. A low exposure of 35 kDa band is provided for clarity. Right panel: 70-kDa band was excised, denatured again, and reduced before re-electrophoresis Actin was the loading control. Nonreduced controls taken at the same exposure are shown adjacent to each blot to highlight antibody specificity, (B): Western blot analysis of secreted STC-1 in conditioned medium. Albumin on the polyvinylidene difluoride membrane was stained with india ink as a loading control. (C): Apoptosis of UV-Fib cultured alone or cocultured with MSCs with or without antibodies to STC-1. p<0.05·Error bars=SD. (D): Fibroblasts were treated with 50 ng/ml or 100 ng/ml for 48 hours following irradiation. Apoptosis was measured using flow cytometry. All experiments shown were performed with MSC donor 1. Abbreviations: Fib, naïve fibroblasts, MSC, multipotent stromal cells; rhSTC-1, recombinant human stanniocalcin 1; STC-1, stanniocalcin-1; UV-Fib, irradiated fibroblasts.

FIG. 3. Upregulation and secretion of STC-1 in MSCs are required for reduction of apoptosis of A549 cells incubated under hypoxia at low pH. (A): Apoptosis of A549 cells. Left panel: A549 cells were incubated alone (light bars) or in coculture with MSC (dark bars) in hypoxia at the pHs indicated. Right panel: Representative flow diagram from pH 5.8, p<0.05. Error bars=SD. (B): Effects of antibodies to STC-1. A549 cells were incubated alone (light bars) or with MSCs (dark bars) at pH 5.8 under hypoxia. Left panel: Apoptosis of A549 cells. Right panel: Apoptosis of MSCs. Antibodies to STC-1 or nonimmune IgG were used at a working dilution of 1:2,000.*, p<0.05. Error bars=SD. (C): Effects of CdM from A549 cells and cocultures CdM was prepared by incubating A549 cells alone (A549 CdM) or in coculture with MSCs (coculture CdM) for 24 hours at pH 5.8 under hypoxia and then transferred to A549 cells incubated under the same conditions for 24 hours with or without addition of antibodies to STC-1 or nonimmune IgG (1:2,000).*, p<0.05. Error bars=SD. (D): Effect of rhSTC. 1 on A549 viability after exposure to hypoxia and low pH for 24 hours. rhSTC-1 was used at a final concentration of 50 ng/ml. Anti-STC-1 was used at a final dilution of 1:1,000.*, p<0.05. Error bars=SD. (E): Knockdown of STC-1 in A549 cells by siRNA.

A549s were co-cultured in transwell with MSCs (MSC transwell), MSCs transfected with a control siRNA (MSC control siRNA), or MSCs transfected with siRNA targeting STC-1 (MSC STC-1 siRNA). Apoptosis was measured using flow cytometry. *, p<0.05. Error bars=SD. All experiments shown were performed with MSC donor 2. Abbreviations: CdM, conditioned medium; MSC, multipotent stromal cells; rhSTC-1, recombinant human stanniocalcin-1; siRNA, short interfering RNA; STC-1, stanniocalcin-1.

FIG. 4. Upregulation and secretion of STC-1 in MSCs are required for reduction of apoptosis of A549 cells incubated under hypoxia at low pH. (A): Western blot for STC-2 in MSC cell lysates when incubated under hypoxia at the indicated pH. Actin was used as a loading control. (B): Western blots for STC-1 in A549 cell lysates when incubated under normoxia and hypoxia at the indicated pH. Note: Panels in (A) and (B) are from the same Western blot taken at the same exposure Norm: pH 7.4 under all conditions Low: pH 6.3 when cells were cultured in normoxic conditions or pH 5.8 under hypoxia. (C): Analysis of conditioned medium for the secreted STC-1 from of A549 cells incubated alone (lanes 4-6) or in coculture with MSCs (lanes 1-3) at the indicated pH in hypoxic conditions. Loading control was albumin. All experiments shown were performed with MSC donor 2. Abbreviations: MSC, multipotent stromal cells; STC-1, stanniocalcin-1.

FIG. 5. STC-1 located at focal adhesions, (A): Cells were fixed with methanol acetone and labeled with antibodies to STC-1. Magnification ×600. (B): Cells fixed with paraformaldehyde and colabeled with antibodies to STC-1 and vinculin, an F-actin binding protein located in focal adhesions. Magnification: top panels, ×600; bottom panels, ×200; inset, ×400. (C): Mouse cells were fixed with methanol/acetone and labeled for STC-1. Magnification, ×200. mIMCD3 cells were negative controls. Magnification ×200; inset, ×400. (D): Left panels: inner medullary collecting duct (IMCD3) cells cocultured directly with MSCs and stained with antibodies to STC-1 and human specific PML. Right panels: IMCD3 cells were treated with rhSTC-1 or cocultured with MSCs in transwell culture and labeled for STC-1 White arrows indicate prominent focal adhesion staining. Magnification, ×200; inset, ×400. For all, levels were adjusted linearly for clarity. Abbreviations: Fib, fibroblast; MEF, mouse embryonic fibroblast mIMCD3 mouse inner medullary collecting duct cell; MSC, multipotent stromal cells; PML, promyelocytic leukemia; rhSTC-1, recombinant human stanniocalcin-1; STC-1, stanniocalcin-1.

FIG. 6. STC-1 location was disrupted in injured cells but preserved when cocultured with MSCs. (A): Fib were fixed with 4% paraformaldehyde and colabeled with antibodies to STC-1 (green) and vinculin (red). Irradiation displaced the STC-1 but not the vinculin from focal adhesions. STC-1 was present in focal adhesions in cocultures. Magnification, ×600. (B): A549 cells were incubated in hypoxia at physiologic or acidic pH, in the absence or presence of MSCs. Cells were stained with antibodies to STC-1. The inset of middle panel is shown with enhanced signal, to display the faint outlines of the cell. Inset of right panel shows distinct focal adhesion staining (arrow). Magnification, upper panels, ×200; insets, ×400, For all, levels were adjusted linearly for clarity. Abbreviations: Fib, fibroblasts; MSC, multipotent stromal cells; STC-1, stanniocalcin-1; UV-Fib, irradiated fibroblasts.

FIG. 7 shows the effect of stanniocalcin-1 (STC-1) on the androgen-dependent growth of LNCaP prostate cancer cells.

FIG. 8 shows the effect of stanniocalcin-1 (STC-1) on the progesterone-dependent growth of T47D breast cancer cells.

FIG. 9 shows that stanniocalcin-1 (STC-1) has no general toxicity to mesenchymal stem cells or PC3 cells.

### DETAILED DESCRIPTION

The present invention relates to the discovery that mesenchymal stromal cells (MSCs) can be manipulated in culture to possess novel therapeutic characteristics and therefore can be useful in therapy of a desired disease. For example, the MSCs can be used to treat diseases associated with apoptosis of cells. This is because the MSCs of the invention can be manipulated to possess therapeutic characteristics including, but not limited to, expressing an anti-apoptotic protein.

Thus, in accordance with an aspect of the present invention, there are provided mesenchymal stem cells which have been cultured under conditions to express an increased amount of at least one anti-apoptotic protein compared to the amount of the at least one anti-apoptotic protein expressed by an otherwise identical population of mesenchymal stem cells which have not been cultured under conditions to express an increased amount of the at least one anti-apoptotic protein.

Anti-apoptotic proteins which may be expressed in increased amounts include, but are not limited to, stanniocalcin-1 (STC-1) and stanniocalcin-2 (STC-2), and any intracellular anti-apoptotic protein.

In a non-limiting embodiment, the anti-apoptotic protein is stanniocalcin-1 (STC-1). In another non-limiting embodiment, the anti-apoptotic protein is stanniocalcin-2 (STC-2).

The present invention provides methods for pre-programming MSCs to express therapeutically beneficial proteins including but not limited to anti-apoptotic proteins prior to transplantation.

In another embodiment the invention provides the use of MSCs to decrease the programmed cell death (apoptosis) that is associated with oxygen deprivation (ischemia and hypoxia) and with multiple kinds of injury to cells and tissues.

In a non-limiting embodiment, skin fibroblasts were UV irradiated to induce apoptosis and the apoptotic fibroblasts were cocultured with MSCs. The MSCs reduced apoptosis of the UV-irradiated fibroblasts. The strategy allowed for the examination of the influence of the apoptotic cells on unperturbed cultures of MSCs. The results presented herein indicate that the MSCs were activated by the apoptotic fibroblasts to upregulate and secrete increased amounts of stanniocalcin-1 (STC-1), a peptide hormone that modulates calcium metabolism and has pleiotrophic effects that include increased resistance of cells to damage from hypoxia and other insults under some circumstances [Westberg et. al., 2007, Stroke 38:1025-103; Westberg et. al., 2007, Am J Physiol Heart Circ Physiol 293:H1766-H1771; Teplova et. al., 2004, Acta Bioch Pol 51:539-544; Koizumi et. al., 2007, Circ J 71:796-801; Ellard et. al., 2007, Mol Cell Endocrinol 264:90-101; Chakraborty et. al., 2007, Am J Physiol 292:F895-F904]. Reduction of apoptosis in the UV-irradiated fibroblasts required STC-1; however, recombinant human STC-1 (rhSTC-1) was unable to reduce apoptosis. In another model, it was observed that MSCs also decreased apoptosis by increased secretion of STC-1 in a coculture system with lung cancer epithelial cells in which both the MSCs and the epithelial cells were exposed to acidosis and hypoxia. Under these circumstances STC-1 was required and sufficient to reduce apoptosis of the lung epithelial cells.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

As used herein, the term “autologous” is meant to refer to any material derived from the same individual to which it is later to be re-introduced into the individual.

As used herein, the term “biocompatible lattice,” is meant to refer to a substrate that can facilitate formation into three-dimensional structures conducive for tissue development. Thus, for example, cells can be cultured or seeded onto such a biocompatible lattice, such as one that includes extracellular matrix material, synthetic polymers, cytokines, growth factors, etc. The lattice can be molded into desired shapes for facilitating the development of tissue types. Also, at least at an early stage during culturing of the cells, the medium and/or substrate is supplemented with factors (e.g., growth factors, cytokines, extracellular matrix material, etc.) that facilitate the development of appropriate tissue types and structures.

As used herein, the term “bone marrow stromal cells,” “stromal cells,” “mesenchymal stem cells,” “mesenchymal stroma cells” or “MSCs” are used interchangeably and refer to a cell derived from bone marrow (reviewed in Prockop, 1997), or other mesenchymal stem cell sources, blood, including peripheral blood (Kuznetsov et. al., 2001), adipose tissue (Guilak et. al., 2004), umbilical cord blood (Rosada et. al., 2003), synovial membranes (De Bari et. al., 2001), periodontal ligament (Seo et. al., 2005), embryonic yolk sac, placenta, umbilical cord, skin, fat, and synovial tissue from joints. Bone marrow cells may be obtained from iliac crest, femora, tibiae, spine, rib, knee, or other mesenchymal tissues. The presence of MSCs in culture colonies may be verified by specific cell surface markers which are identified with monoclonal antibodies. See U.S. Pat. Nos. 5,486,359 and 7,153,500. MSCs are characterized by their ability to adhere to plastic tissue culture surfaces (Friedenstein et. al., reviewed in Owen & Friedenstein, 1988), and by being an effective feeder layers for hematopoietic stem cells (Eaves et. al., 2001). In addition, MSCs can be differentiated both in culture and in vivo into osteoblasts and chondrocytes, into adipocytes, muscle cells (Wakitani et. al., 1995) and cardiomyocytes (Fukuda and Yuasa, 2006), into neural precursors (Woodbury et. al., 2000; Deng et. al., 2001, Kim et. al., 2006; Mareschi et. al., 2006; Krampera et. al., 2007), and into bone, cartilage, ligament, tendon, cardiac tissue, stroma, dermis, and other connective tissues. (See U.S. Pat. Nos. 6,387,369 and 7,101,704.). Mesenchymal stem cells (MSCs) may be purified using methods known in the art (Wakitani et. al., 1995; Fukuda and Yuasa, 2006; Woodbury et al., 2000; Deng et. al., 2001; Kim et. al., 2006; Mareschi et. al., 2006; Krampera et. al., 2007).

“Graft” refers to a cell, tissue, organ or otherwise any biological compatible lattice for transplantation.

“Allogeneic” refers to a graft derived from a different animal of the same species.

“Xenogeneic” refers to a graft derived from an animal of a different species.

“Transplant” refers to a biocompatible lattice or a donor tissue, organ or cell, to be transplanted. An example of a transplant may include but is not limited to skin cells or tissue, bone marrow, and solid organs such as heart, pancreas, kidney, lung and liver. Preferably, the transplant is a human neural stem cell.

As defined herein, an “allogeneic bone marrow stromal cell (BMSC)” is obtained from a different individual of the same species as the recipient.

“Donor antigen” refers to an antigen expressed by the donor tissue to be transplanted into the recipient.

“Alloantigen” is an antigen that differs from an antigen expressed by the recipient.

As used herein, an “effector cell” refers to a cell which mediates an immune response against an antigen. In the situation where a transplant is introduced into a recipient, the effector cells can be the recipient's own cells that elicit an immune response against an antigen present in the donor transplant. In another situation, the effector cell can be part of the transplant, whereby the introduction of the transplant into a recipient results in the effector cells present in the transplant eliciting an immune response against the recipient of the transplant.

As used herein, a “therapeutically effective amount” is the amount of MSCs which is sufficient to provide a beneficial effect to the subject to which the MSCs are administered.

As used herein “endogenous” refers to any material from or produced inside an organism, cell or system.

“Exogenous” refers to any material introduced from or produced outside an organism, cell, or system.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### Description

The present invention, in a non-limiting embodiment, relates to the discovery that when MSCs are contacted with an apoptotic cell, the MSCs express high levels of at least one anti-apoptotic protein, including but not limited to, STC-1. The disclosure presented herein demonstrates that MSCs contacted with an apoptotic cell express increased amounts of at least one anti-apoptotic protein, including but not limited to, STC-1, thereby exhibiting an anti-apoptotic property(ies).

In another non-limiting embodiment, the mesenchymal stem cells which express increased amounts of at least one anti-apoptotic protein are cultured under conditions which provide for the aggregation of the mesenchymal stem cells into a spheroidal aggregate.

The present invention encompasses methods and compositions for inhibiting, preventing, reducing and/or eliminating apoptosis in a mammal by treating the mammal with an effective amount of MSCs that express increased amounts of at least one anti-apoptotic protein.

The present invention provides methods of pre-programming MSCs to express therapeutically beneficial anti-apoptotic proteins prior to transplantation into a recipient. In some instances, the method includes activating MSCs and administering MSCs so that anti-apoptotic proteins are at maximal expression when administrated to a patient.

### Therapy to Inhibit Apoptosis

The present invention includes a method of using MSCs that have been cultured under conditions to express increased amounts of at least one anti-apoptotic protein as a therapy to inhibit or prevent apoptosis. In a non-limiting embodiment, the MSCs which are used as a therapy to inhibit or prevent apoptosis have been contacted with an apoptotic cell. The invention is based on the discovery that MSCs that have been contacted with an apoptotic cell express high levels of anti-apoptotic molecules. In some instances, the MSCs that have been contacted with an apoptotic cell secrete high levels of at least one anti-apoptotic protein, including but not limited to, STC-1.

One skilled in the art would appreciate, based upon the disclosure provided herein, that the ability of MSCs that have been contacted with an apoptotic cell to suppress apoptosis provides a means for an anti-apoptosis therapy.

Based upon the disclosure provided herein, MSCs can be obtained from any source. The MSCs may be autologous with respect to the recipient (obtained from the same host) or allogeneic with respect to the recipient. In addition, the MSCs may be xenogeneic to the recipient (obtained from an animal of a different species); for example, rat MSCs may be used to suppress apoptosis in a human.

In a further non-limiting embodiment, MSCs used in the present invention can be isolated, from the bone marrow of any species of mammal, including but not limited to, human, mouse, rat, ape, gibbon, bovine. In a non-limiting embodiment, the MSCs are isolated from a human, a mouse, or a rat. In another non-limiting embodiment, the MSCs are isolated from a human.

Based upon the present disclosure, MSCs can be isolated and expanded in culture in vitro to obtain sufficient numbers of cells for use in the methods described herein provided that the MSCs are cultured in a manner that promotes contact with an apoptotic cell. For example, MSCs can be isolated from human bone marrow and cultured in complete medium (DMEM low glucose containing 4 mM L-glutamine, 10% FBS, and 1% penicillin/streptomycin) in hanging drops or on non-adherent dishes. The invention, however, should in no way be construed to be limited to any one method of isolating and/or to any culturing medium. Rather, any method of isolating and any culturing medium should be construed to be included in the present invention provided that the MSCs are cultured in a manner that provides MSCs to express increased amounts of at least one anti-apoptotic protein.

Any medium capable of supporting MSCs in vitro may be used to culture the MSCs. Media formulations that can support the growth of MSCs include, but are not limited to, Dulbecco's Modified Eagle's Medium (DMEM), alpha modified Minimal Essential Medium (αMEM), and Roswell Park Memorial Institute Media 1640 (RPMI Media 1640) and the like. Typically, up to 20% fetal bovine serum (FBS) or 1-20% horse serum is added to the above medium in order to support the growth of MSCs. A defined medium, however, also can be used if the growth factors, cytokines, and hormones necessary for culturing MSCs are provided at appropriate concentrations in the medium. Media useful in the methods of the invention may contain one or more compounds of interest, including, but not limited to, antibiotics, mitogenic or differentiation compounds useful for the culturing of MSCs. The cells may be grown at temperatures between 27° C. to 40° C., preferably 31° C. to 37° C., and more preferably in a humidified incubator. The carbon dioxide content may be maintained between 2% to 10% and the oxygen content may be maintained between 1% and 22%. The invention, however, should in no way be construed to be limited to any one method of isolating and culturing MSCs. Rather, any method of isolating and culturing MSCs should be construed to be included in the present invention.

Antibiotics which can be added into the medium include, but are not limited to, penicillin and streptomycin. The concentration of penicillin in the culture medium, in a non-limiting embodiment, is about 10 to about 200 units per ml. The concentration of streptomycin in the culture medium is, in a non-limiting embodiment, about 10 to about 200 μg/ml.

The mesenchymal stem cells which express increased amounts of at least one anti-apoptotic protein may be administered to an animal in an amount effective to provide a therapeutic effect. The animal may be a mammal, including but not limited to, human and non-human primates.

Another embodiment of the present invention encompasses the route of administering MSCs to the recipient of the transplant. MSCs can be administered by a route which is suitable for the placement of the transplant, i.e. a biocompatible lattice or a donor tissue, organ or cell, to be transplanted. MSCs can be administered systemically, i.e., parenterally, by intravenous injection or can be targeted to a particular tissue or organ, such as bone marrow, or MSCs can be administered via a subcutaneous implantation of cells or by injection of the cells into connective tissue, for example, muscle.

The MSCs can be suspended in an appropriate diluent. Suitable excipients for injection solutions are those that are biologically and physiologically compatible with the MSCs and with the recipient, such as buffered saline solution or other suitable excipients. The composition for administration can be formulated, produced, and stored according to standard methods complying with proper sterility and stability.

The MSCs may have one or more genes modified or be treated such that the modification has the ability to cause the MSCs to self-destruct or “commit suicide” because of such modification, or upon presentation of a second drug (eg., a prodrug) or signaling compound to initiate such destruction of the MSCs.

The dosage of the MSCs varies within wide limits and may be adjusted to the individual requirements in each particular case. The number of cells used depends on the age, weight, sex, and condition of the recipient, the number and/or frequency of administrations, the disease or disorder being treated, and the extent or severity thereof, and other variables known to those of skill in the art.

In a non limiting embodiment, the MSCs may be administered in combination with other drugs which possess anti-apoptotic activity.

### Advantages of Using MSCs

Based upon the disclosure herein, it is envisioned that the MSCs of the present invention can be used in conjunction with current modes, for example the use of anti-apoptotic therapy, for the treatment of diseases, disorders, or conditions associated with apoptosis. An advantage of using MSCs in place of or in conjunction with anti-apoptotic agents is that by using the methods of the present invention to ameliorate the severity of apoptosis in the recipient, the amount of anti-apoptotic agents used and/or the frequency of administration of anti-apoptotic agents can be reduced. A benefit of reducing the use of anti-apoptotic agents is the alleviation of unwanted side effects associated with anti-apoptotic agents.

It is also contemplated that the cells of the present invention may be administered into a recipient as a “one-time” therapy for the treatment of apoptosis. A one-time administration of MSCs into the recipient eliminates the need for chronic anti-apoptotic therapy; however, if desired, multiple administrations of MSCs may also be employed.

The invention described herein also encompasses a method of preventing or treating apoptosis by administering MSCs in a prophylactically or therapeutically effective amount for the prevention, treatment, or amelioration of apoptosis. An effective amount of MSCs can be determined by comparing the level of apoptosis in a recipient prior to the administration of MSCs thereto, with the level of apoptosis present in the recipient following the administration of MSCs thereto. A decrease, or the absence of an increase, in the level of apoptosis in the recipient with the administration of MSCs thereto, indicates that the number of MSCs administered is a therapeutically effective amount of MSCs.

Applicants also have discovered that STC-1 represses reactive oxygen species (ROS) production in prostate cancer cells and in breast cancer cells. The ROS production is mediated by p66Shc protein, and such ROS production is required for growth of such cancer cells. Thus, in accordance with another aspect of the present invention, there is provided a method of treating a cancer which is responsive to anti-apoptotic proteins or a cancer in which disruption of reactive oxygen species results in anti-cancer activity, by administering to a patient at least one anti-apoptotic protein or cells which express at least one anti-apoptotic protein. The at least one anti-apoptotic protein and/or cells which express at least one anti-apoptotic protein is/are administered in an amount effective to treat the cancer which is responsive to anti-apoptotic proteins, or the cancer in which disruption of anti-apoptotic proteins results in anti-cancer activity.

Cancers which may be treated include, but are not limited to, prostate cancer, breast cancer, and any other cancer which is responsive to at least one anti-apoptotic protein, or any other cancers in which disruption of reactive oxygen species results in anti-cancer activity.

The at least one anti-apoptotic protein may be obtained by means known to those skilled in the art, such as by purification, production by recombinant means, or may be synthesized on an automatic protein synthesizer. Alternatively, in a non-limiting embodiment, one may administer cells which express, either naturally or recombinantly, the at least one anti-apoptotic protein. In a non-limiting embodiment, the cells are mesenchymal stem cells. In yet another non-limiting embodiment, the mesenchymal stem cells have been cultured under conditions whereby the mesenchymal stem cells express increased amounts of the at least one anti-apoptotic protein, as hereinabove described.

In a non-limiting embodiment, the at least one anti-apoptotic protein is STC-1.

Thus, based upon the disclosure herein, it is envisioned that the MSCs of the present invention can be used alone, or in conjunction with current modes, for example, the use of anti-tumor therapy, for the treatment of cancers which are responsive to anti-apoptotic proteins, or cancers in which disruption of reactive oxygen species results in anti-cancer activity. Cancers which may be treated by the mesenchymal stem cells of the present invention include, but are not limited to, prostate cancer, breast cancer, and any other cancer which is responsive to anti-apoptotic proteins, or cancers in which disruption of reactive oxygen species results in anti-cancer activity. An advantage of using MSCs in place of or in conjunction with anti-tumor agents is that by using the methods of the present invention to ameliorate the severity of the above-mentioned cancers in the recipient, the amount of anti-tumor agents used and/or the frequency of administration of anti-tumor agents can be reduced. A benefit of reducing the use of anti-tumor agents is the alleviation of unwanted side effects associated with anti-tumor agents.

It is also contemplated that the cells of the present invention may be administered into a recipient as a “one-time” therapy for the treatment of cancers which are responsive to anti-apoptotic proteins, or in which the disruption of reactive oxygen species results in anti-cancer activity. A one-time administration of MSCs into the recipient eliminates the need for chronic anti-tumor therapy for such cancers. If desired, however, multiple administrations of MSCs may also be employed.

The invention described herein also encompasses a method of preventing or treating cancers that are responsive to anti-apoptotic proteins, or cancers in which the disruption of reactive oxygen species results in anti-cancer activity, by administering MSCs in a prophylactically or therapeutically effective amount for the prevention, treatment or amelioration of cancers that are responsive to anti-apoptotic proteins, or cancers in which the disruption of reactive oxygen species results in anti-cancer activity. An effective amount of MSCs can be determined by comparing the level of a cancer which is responsive to anti-apoptotic proteins, or cancers in which the disruption of reactive oxygen species results in anti-cancer activity, in a recipient prior to the administration of MSCs thereto, with the level of the cancer which is responsive to anti-apoptotic proteins present, or the level of a cancer in which the disruption of reactive oxygen species results in anti-cancer activity present, in the recipient following the administration of MSCs thereto. A decrease, or the absence of an increase, in the level of the cancer which is responsive to anti-apoptotic proteins, or in the level of the cancer in which the disruption of reactive oxygen species results in anti-cancer activity, in the recipient with the administration of MSCs thereto, indicates that the number of MSCs administered is a therapeutic effective amount of MSCs.

In one non-limiting embodiment, the mesenchymal stem cells which express increased amounts of at least one anti-apoptotic protein, such as STC-1 or STC-2, for example, are administered to a cancer patient in order to prevent, limit, or treat damage to non-cancerous cells, which may be caused by anti-cancer treatments such as chemotherapy and radiation, for example. The mesenchymal stem cells, in a non-limiting embodiment, may be administered in combination with other agents which prevent, limit, or treat damage to non-cancerous cells.

In a non-limiting embodiment, the mesenchymal stem cells which express increased amounts of at least one anti-apoptotic protein may be administered in conjunction with at least one protein that induces apoptosis of cancer cells. Such proteins that induce apoptosis of cancer cells, however, also may induce undesired apoptosis of non-cancerous cells. Thus, the mesenchymal stem cells which express increased amounts of at least one anti-apoptotic protein are administered in conjunction with at least one protein that induces apoptosis of cancer cells in order to prevent, limit, or treat damage to non-cancerous cells which may be caused by the protein that induces apoptosis of cancer cells.

The mesenchymal stem cells which express increased amounts of the at least one anti-apoptotic protein may be administered prior to, subsequent to, or concurrently with the at least one protein which induces apoptosis of cancer cells.

The protein which induces apoptosis of cancer cells may be obtained by means known to those skilled in the art, such as by purification, production by recombinant means, or may be synthesized by an automatic protein synthesizer. Alternatively, in a non limiting embodiment, one may administer, in conjunction with the mesenchymal stem cells which express increased amounts of the at least one anti-apoptotic protein, cells which express, either naturally or recombinantly, the at least one protein which induces apoptosis of cancer cells. In a non-limiting embodiment, the cells are mesenchymal stem cells.

In another non-limiting embodiment, the at least one apoptosis protein that induces apoptosis of cancer cells is tumor necrosis factor-related apoptosis-inducing ligand, or TRAIL.

Thus, in a non-limiting embodiment, the mesenchymal stem cells which express increased amounts of at least one anti-apoptotic protein are administered to a patient in conjunction with TRAIL, or a cell which expresses TRAIL, whereby one prevents, limits, or inhibits the growth of cancer cells while preventing, limiting, or inhibiting damage to non-cancerous cells.

In another non-limiting embodiment, the mesenchymal stem cells which express increased amounts of at least one anti-apoptotic protein are administered to treat damage to cells caused by radiation poisoning. In one non-limiting embodiment, the cells which have been damaged by radiation poisoning are non-cancerous cells which have been damaged by radiation employed in an anti-cancer treatment. In another non-limiting embodiment, the radiation poisoning is a result of prolonged and/or unprotected exposure to one or more sources of radiation or radioactive materials. Such sources of radiation or radioactive materials include, but are not limited to, x-rays, gamma-rays, materials used as fuel for nuclear power plants, nuclear waste, and materials employed in nuclear medicine, either as therapeutic or diagnostic agents.

Based upon the disclosure herein, it is envisioned that the MSCs of the present invention can be used in conjunction with current modes, for example the use of immune modulation therapy for graft versus host disease following transplants of bone marrow or organs or for auto-immune diseases such as lupus and autoimmune related diseases such as Type 1 diabetes, rheumatoid arthritis, thyroiditis, and psoriasis, and autoproliferative diseases, as well as atherosclerosis.

Based upon the disclosure herein, it is envisioned that the MSCs of the present invention can be used in conjunction with current modes, for example the use of therapies to limit programmed cell death as occurs following injury to tissues or organs from lack of oxygen (ischemia or hypoxia), such as, for example, myocardial infarction, and limb ischemia, or in diseases such as Alzheimer's disease, parkinsonism, Down's Syndrome, and other neurodegenerative diseases as well as stroke, brain trauma, or concussion, multiple sclerosis, or muscular dystrophy.

Although the scope of the present invention is not to be limited to any theoretical reasoning, unresolved inflammation now is recognized to be part of the pathology of a series of chronic diseases that include, but is not limited to, diabetes, parkinsonism, and Alzheimer's disease, as well as when ischemia (a lack of oxygen that damages mitochondria) is followed by restoration of blood.

Cells must have a means of reducing oxygen to water; however, the process frequently is inefficient and gives rise to partially reduced and highly reactive forms of oxygen referred to as reactive oxygen species, or ROS. The unresolved inflammation is caused in part by an increase in reactive oxygen species. STC-1 recently has been shown to reduce reactive oxygen species by increasing expression of uncoupling protein-2, that makes mitochondria more efficient in reducing ROS. Therefore, cells which express at least one anti-apoptotic protein, including the MSCs of the present invention, which express increased amounts of at least one anti-apoptotic protein, may be useful in reducing reactive oxygen species and unresolved inflammation in the above-mentioned diseases and conditions. Such cells thus also may be anti-inflammatory as well as anti-apoptotic.

Thus, in accordance with yet another aspect of the present invention, there is provided a method of treating a disease or disorder that is associated with excessive production of ROS in an animal (including human and non-human animals) by administering an effective amount of at least one anti-apoptotic protein. In one non-limiting embodiment, the at least one anti-apoptotic protein is STC-1.

Diseases or disorders which are associated with excessive production of ROS, and which may be treated, include, but are not limited to, autoimmune diseases such as lupus, Type I diabetes, rheumatoid arthritis, thyroiditis, and psoriasis, and autoproliferative diseases, atherosclerosis, myocardial infarction, ischemia or hypoxia, including limb ischemia, Alzheimer's disease, parkinsonism, Down's Syndrome, and other neurodegenerative diseases, as well as stroke, brain trauma, concussion, multiple sclerosis, or muscular dystrophy, as well as when ischemia is followed by restoration of blood, and any disease or disorder associated with unresolved inflammation.

The mesenchymal stem cells of the present invention also may be used to treat or prevent organ death and/or loss of organ function resulting from loss of normal blood flow to the organ, or from infection and/or shock including septic shock. In a non-limiting embodiment, the mesenchymal stem cells of the present invention may be used to treat ischemia in the heart following a heart attack, especially where reperfusion to the heart causes cell apoptosis.

The mesenchymal stem cells of the present invention also may be used to treat or prevent cell apoptosis, and/or cell death resulting from the introduction of one or more toxins into a patient, such as toxins introduced as a result of a bite or from other toxin-transferring contact with a venomous animal (eg., snake, lizard, insect, arachnid, fish, jellyfish), for example.

The following examples further illustrate aspects of the present invention; however, they are in no way a limitation of the teachings or disclosure of the present invention as set forth herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

Multipotent stromal cells (MSCs) have been shown to reduce apoptosis in injured cells by secretion of paracrine factors, but these factors were not fully defined. We observed that coculture of MSCs with previously UV-irradiated fibroblasts reduced apoptosis of the irradiated cells, but fresh MSC conditioned medium was unable reproduce the effect. Comparative microarray analysis of MSCs grown in the presence or absence of UV-irradiated fibroblasts demonstrated that the MSCs were activated by the apoptotic cells to increase synthesis and secretion of stanniocalcin-1 (STC-1), a peptide hormone that modulates mineral metabolism and has pleiotrophic effects that have not been characterized fully. We showed that STC-1 was required but not sufficient for reduction of apoptosis of UV-irradiated fibroblasts. In contrast, we demonstrated that MSC-derived STC-1 was both required and sufficient for reduction of apoptosis of lung cancer epithelial cells made apoptotic by incubation at low pH in hypoxia. Our data demonstrate that STC-1 mediates the anti-apoptotic effects of MSCs in two distinct models of apoptosis in vitro.

The materials and methods employed in the experiments disclosed herein are now described.

### Cell Culture and Reagents

Frozen vials of passage 1 human bone marrow MSCs (approximately 1×10⁶ cells) were obtained from Tulane University (http://www.som.tulane.edu/gene_therapydistribute.shtml)

The cells differentiated consistently into bone, fat, and cartilage in culture and were negative for hematopoietic markers (CD34, CD36, CD117, and CD45) and positive for CD29 (95%), CD44 (>93%), CD49c (99%), CD49f (>70%), CD59 (>99%), CD90 (>99%), CD 105 (>99%), and CD 166 (>99%). The cells were thawed and plated in a 15-cm-diameter dish in complete culture medium (CCM) (α-minimal essential medium; Gibco-BRL, Carlsbad, Calif., http://www.gibcobrl.com), 17% fetal bovine serum (FBS; lot-selected for rapid growth of MSCs; Atlanta Biologicals, Atlanta, Ga., http://www.atlantabio.com), 100 units/ml penicillin/100 mg/ml streptomycin/2 mM L-glutamine (Gibco-BRL) and incubated for 24 hours to recover viable cells [Sekiya et al., 2002, Stem Cells 20:530.-541]. The medium was removed, the cultures were washed with phosphate-buffered saline (PBS), and adherent MSCs were recovered by incubation with 0.25% trypsin and 1 mM EDTA (Gibco-BRL) for 5 minutes at 37° C. Donors used were as follows: donor 1, 5064L; donor 2, 240L; donor 3, 242L.

For the coculture experiments with irradiated fibroblasts, normal human diploid dermal skin fibroblasts (HS-68; American Type Culture Collection, Rockville, Md., http://www.atcc.org) were thawed and plated at 10,000 cells per cm² on a 4.6-cm² transwell inserts (pore size, 0.4 μm; #3450; Corning Enterprises, New York, http://www.corning.com) in 2 ml of growth medium (low-glucose Dulbecco's modified Eagle's medium [Gibco-BRL], 10% FBS, and 100 units/ml penicillin). Cells were incubated for 24 hours and then irradiated with 50 J/m² UV light (Stratalinker model 1800; Stratagene, Santa Clara, Calif., http://www.stratagene.com). This amount of UV light was determined to be optimal for achieving apoptosis in 15%-30% of the cells in 48 hours. Irradiated fibroblasts were incubated in coculture by placing the filters over MSCs that were previously plated at a density of 1,000 cells per cm² on regular six-well dishes (#3516; Corning, N.Y., N.Y.) and incubated for 5 days, with a medium change on the third day. Cocultures and controls were incubated in 3 ml of CCM for 48 hours. The fibroblasts were then harvested with trypsin/EDTA.

For the coculture experiments with human A549 lung epithelial cells (American Type Culture Collection), the A549 cells were plated at 10,000 cells per cm² on transwell inserts and incubated in growth medium for 24 hours. Inserts were then placed on top of MSCs that were previously plated at 1,000 cells per cm² and were preincubated for 5 days in 3 ml of CCM. The CCM was either unmodified or preadjusted to a predetermined pH with lactic acid (Sigma-Aldrich, St. Louis, http://www.sigmaadrich.com). For hypoxia experiments, A549 and MSC cocultures were incubated under 1% O₂, 5% CO₂, and 94% N₂ for 24 hours (model 3130 incubator; Thermo Electron Corporation, Holsbrook, N.Y., http://www.thermo.com). After a 24-hour incubation period, cells were harvested. pH 5.8 was found to be optimal to induce apoptosis under hypoxia, whereas pH 6.3 was optimal under normoxia.

Mouse embryonic fibroblasts from wild-type and STC-1-overexpressing mice were a gift from Dr. Gabriel DiMattia (University of Western Ontario). Inner medullary collecting duct cells (IMCD3 cells) were a gift from Dr. Samir El-Dahr (Tulane University Health Sciences Center).

Donkey anti-STC-1 antibodies from two sources (R&D Systems Inc., Minneapolis, http://www.rndsystems.com; Santa Cruz Biotechnology Inc., Santa Cruz, Calif., http://www.scbt.com) or an isotype control of donkey anti-IgG (Beckman Coulter, Brea, CA, http://www.beckmancoulter.com) was added to the medium at the indicated concentrations. FLAG-tagged rhSTC-1 synthesized in human cells was purchased from Bio Vendor Laboratory Medicine, Inc. (Modrice, Czech Republic, http://www.biovendor.com).

### Viability Assays

Viability was assayed with annexin V-fluorescein isothiocyanate and propidium iodide (PI) (Annexin V-F1TC Apoptosis Detection Kit; Sigma-Aldrich) and analyzed with a closed-stream flow cytometer (model FCSOO; Beckman Coulter). Photomicrographs were prepared by phase contrast microscopy (Eclipse TE200; Nikon, Tokyo, http://www.nikon.com). Cell cycle analysis was performed using the DNA-Prep Reagent System (Beckman Coulter) and analyzed by flow cytometry as described previously [Larson et. al., 2008, Stem Cells 26:193-201]. Terminal deoxynucleotidyl transferase dUTP nick-end labeling (TUNEL) staining was performed using the Roche In Situ Cell Death Detection Kit (Roche Diagnostics, Indianapolis, http://www.roche-applied-science.com) as per the manufacturer's instructions. Mitochondria were stained using the MitoTracker Red CM-H2XRos mitochondrial dye as per the manufacturer's instructions (Invitrogen, Carlsbad, Calif., http://www.invitrogen.com).

### Microarrays

To obtain adequate amounts of RNA for the microarrays, the coculture experiments were repeated with fibroblasts that were plated at 10,000 cells per cm² on a 9.6-cm² transwell permeable 0.4-μm pore filter (Corning) for 24 hours and then UV-irradiated. The transwell filter was cocultured with MSCs by placing the filter over MSCs that were previously plated at 100 cells per cm² in a 15-cm² dish and incubated for 5 days. The transwell filter was supported over the MSCs with 3×3 mm pieces of sterile silicone that were 1 mm thick (Press-to-Seal; Invitrogen). The samples were incubated in 30 ml of CCM for 48 hours. The MSCs were lysed, and RNA was isolated (RNeasy RNA extraction kit; Qiagen, Valencia, Calif., http://wwwl.qiagen.com) RNA concentration was assayed by absorbance at 260 nm. Samples were processed by the Microarray Core Facility of the Tulane Center for Gene Therapy [Ylostalo et. al., 2006, Stem Cells 24:642-652]. In brief, microarrays were performed using a GeneChip (HGU1332.0; Affymetrix, Santa Clara, Calif., http://www.affymetrix.com) for 55,000 human probes for transcripts from more than 30,000 human genes. Chips were scanned with Microarray Suite 5.0 (MAS5.0; Affymetrix), and the images were transferred to the dChipl.3+program [Li et al., 2001, Proc Natl Acad Sci USA 98:31-36]. A heat map was generated by clustering genes upregulated or downregulated more than twofold, at 90% confidence.

### Western Blot Assay

Cells were lysed (RIPA Lysis Buffer; Santa Cruz Biotechnology) and suspended in sample buffer (NuPAGE LDS sample buffer; Invitrogen) containing 5% 2-mercaptoethanol (Sigma-Aldrich), heated for 3 minutes at 95° C., and loaded at 20 μg of protein per lane onto polyacrylamide gels (NuPAGE 4%-12% Bis-Tris Gels; Invitrogen). Electrophoresis was for 1.5 hours at 180 V in running buffer (NuPAGE MOPS SDS Running Buffer; Invitrogen). Polyvinylidene difluoride (PVDF) membrane (GenHunter Corporation, Nashville, Tenn., http://www.genhunter.com) was incubated in methanol for 1 minute, and proteins were transferred to the membrane by electrophoresis at 30 V for 1.5 hours in transfer buffer (NuPAGE Transfer Buffer; Invitrogen). The membrane was blocked for 2 hours at room temperature with PBS containing 0.5% Tween 20 (PBST) and 5% skim milk (Santa Cruz Biotechnology). The membrane was incubated overnight at 4° C. with-primary goat antibody to STC-1(1:1,000; R&D Systems) in 1% skim milk. After washing with PBST, the membrane was incubated for 2 hours at room temperature with secondary horseradish peroxidase-conjugated donkey anti-goat antibody (1:5,000; Millipore, Temecula, Calif. http://www.millipore.com) in 1% skim milk in PBST. The membrane was visualized by chemiluminescence (Visualizer Spray & Glow ECL Western Blotting Detection System; Upstate, Lake Placid, N.Y., http://www.upstate.com). To isolate the 70-kDa band of STC-1, the corresponding region of the gel was excised using the molecular weight standard as a guide. The excised gel was placed in dialysis tubing and was electrophoresed for 1 hour to elute the protein in 5 ml of 0.1× running buffer (NuPAGE MOPS SDS running buffer; Invitrogen). The sample was then lyophilized (MODULYOD; Thermo Electron Corporation) and resuspended in 100 μl of lysis buffer (RIPA lysis buffer; Santa Cruz Biotechnology). Ten microliters of sample, containing 1× loading buffer (NuPAGE LDS sample buffer; Invitrogen) and 5% 2-mercaptoethanol, was boiled for 3 minutes and electrophoresed in 0.1× running buffer (NuPAGE MOPS SDS running buffer; Invitrogen). For assays of secreted STC-1, conditioned medium was passed through a 50-kDa filter (Amicon Ultra Centrifugal Filter; Millipore), and the filtrate was concentrated on a 10-kDa filter. The concentrated sample was then assayed by electrophoresis and Western blotting. Equal loading of protein was confirmed by staining the PVDF membrane with india ink (Pelikan, Hannover, Germany).

### Quantitative Reverse Transcription-Polymerase Chain Reaction

Cells were lysed, RNA was isolated (RNeasy RNA extraction kit; Qiagen), and RNA concentration was assayed by absorbance at 260 nm. Reverse transcription was carried out (Superscript III; Invitrogen), and quantitative real-time polymerase chain reaction (PCR) was performed (ABI Prism 7700 Sequence Detection System using a SYBR green kit; Applied Biosystems, Foster City, Calif., http://www.appliedbiosystems.com) using the following primer pairs: STC-1 forward, CAG CTG CCC AAT CAC TTC TC (SEQ ID NO: 1); STC-1 reverse, TCT CCA TCA GGC TGT CTC TGA (SEQ ID NO: 2); glyceraldehyde-3-phosphate dehydrogenase (GAPDH) forward, TCA ACG GAT TTG GTC GTA TTG GG (SEQ ID NO: 3); GAPDH reverse, TGA TTT TGG AGG GAT CTC GC (SEQ ID NO: 4).

### RNA Interference and Transfection

Three different short interfering RNAs (siRNAs) for STC-1 (Silencer Pre-designed siRNA; catalog no. AM16708A; Ambion, Austin, Tex., http://www.ambion.com) were used with a negative control (Silencer FAMTM-Labeled Negative Control 1 siRNA; catalog no. AM4620; Ambion). siRNA transfection was carried out using a commercial kit (siPORT Neo FX; Ambion). Briefly, 5 μl of SiPORT Neo FX in 100 μl of Opti-MEM (Invitrogen) was mixed with either STC-1 100 nM siRNA or 300 nM negative control in 100 μl of Opti-MEM, The mixture was then incubated for 20 minutes at room temperature and was added to freshly trypsinized MSCs in suspension (5,000 cells per 800 μl of CCM). Final siRNA concentration was 30 nM. The cells were incubated for 16 hours at 37° C. in a 5% CO₂ incubator and were used for experiments within 48 hours. Following incubation, transfection efficiency was evaluated using flow cytometry. STC-1 knockdown was confirmed by Western blotting and reverse transcription-PCR. The following three siRNA probes were used simultaneously to knock down STC-1: ID12722 sense, GGG AA AAGCAUUCGUCAAAtt (SEQ ID NO: 5); antisense, UUUGACGAAUGCUUUU CCCtg (SEQ ID NO: 6); ID12905 sense, GGUCUAACUGUGGAAUAUAtt (SEQ ID NO: 7); antisense, UAUAUUCCACAGUUAGACCtt (SEQ ID NO: 8); ID138790 sense, CGA CUAACCUAUCUAUGAAtt (SEQ ID NO: 9); antisense, UUCAUAGAUAGGUU AGUCGtt (SEQ ID NO: 10).

### Immunofluorescence Microscopy

Cells grown on coverslips were fixed for 10 minutes either with ice-cold methanol acetone (1:1) for labeling with antibodies to STC-1 or with 4% paraformaldehyde (Electron Microscopy Science, Hatfield, Pa., http://www.emsdiasum.com) for labeling with antibodies to both STC-1 and vinculin. The sample was washed three times for 5 minutes with PBS, blocked for 45 minutes at room temperature in 5% donkey serum in PBS, and incubated for 1 hour at room temperature in donkey anti-STC-I antibody (1:1,000; R&D Systems). For experiments marked “data not shown,” anti-STC-1 antibody was obtained from Santa Cruz Biotechnology. The sample was washed three times for 5 minutes with PBS and incubated for 1 hour with Alexa-594-conjugated donkey anti-goat IgG (1:1,000; invitrogen). After three 5-minute washes in PBS, coverslips were mounted on slides. For vinculin/STC-1 double labeling, primary antibody solution also contained mouse anti-vinculin antibody (1:500; Abcam, Cambridge, Mass., http://www.abcam.com) and an additional secondary (Alexa-488 donkey anti-mouse). Rabbit anti-promyelocytic leukemia (PML) antibody (Millipore) was used at a final concentration of 1:1,000. Anti-rabbit Alexa-488 conjugated antibody (Invitrogen) was used at a final concentration of 1:1,000. Photomicrographs were obtained with an epifluorescence microscope (model BX51; Olympus, Tokyo, http://www.olympus-global.com) with an ORCA-AG digital charge-coupled device camera (Hamamatsu Photonics, Hamamatsu, Japan, http://www.hamamatsu.com) A nonspecific goat IgG (Santa Cruz Biotechnology) was used as a negative control. For absorption assays, antibodies were incubated with 50 ng/ml rhSTC-1 45 minutes prior to incubation with sample. Surface plot rendering of STC-1 staining was performed using ImageJ (http://rsb.info.nih.gov/ij).

### Transient Transfection of A549 Cells

Complimentary DNA corresponding to STC-1 was obtained from American Tissue Type Culture Collection. Approximately 1×10⁵ A549 cells were plated on 22×22 mm glass coverslips in a six-well dish and transfected using Trans-it LT-1 transfection reagent (Minis Bio, Madison, Wis., http://www.mirusbio.com). Briefly, 1 μg of STC-1 DNA was mixed with 1.5 μg of carrier DNA (Bluescript SK+), incubated with 7.5 μl of Trans-it LT-1 reagent diluted in 250 μl of Opti-MEM, and transferred into a well containing 2 ml of growth medium.

### Statistical Analyses

Unless otherwise indicated, all experiments were performed in triplicate, a minimum of three times. Analysis of variance was performed for experiments of more than two groups; otherwise, a two-tailed, unpaired Student t test was performed. Statistical analysis was processed using Smith's Statistical Package (http://www.economics.pomona.edu/statite/ssp.html).

The results of the experiments disclosed herein are now described.

### MSCs Reduce Apoptosis of UV-Irradiated Fibroblasts

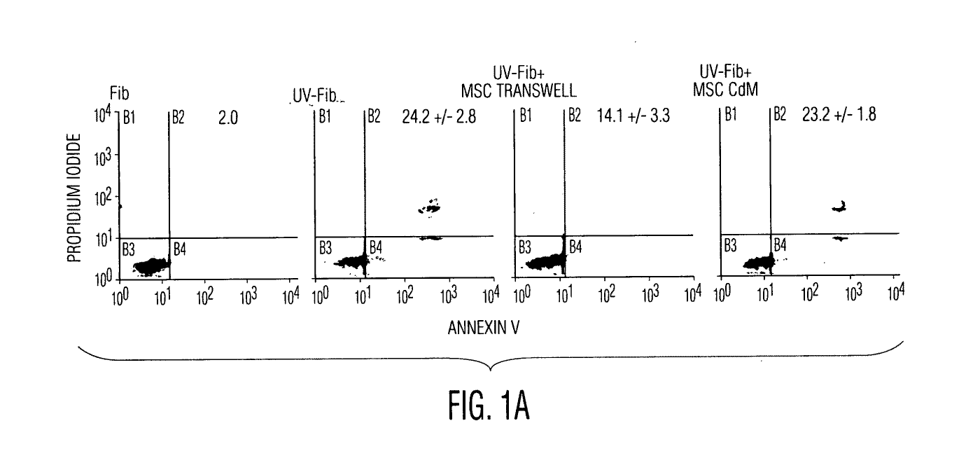

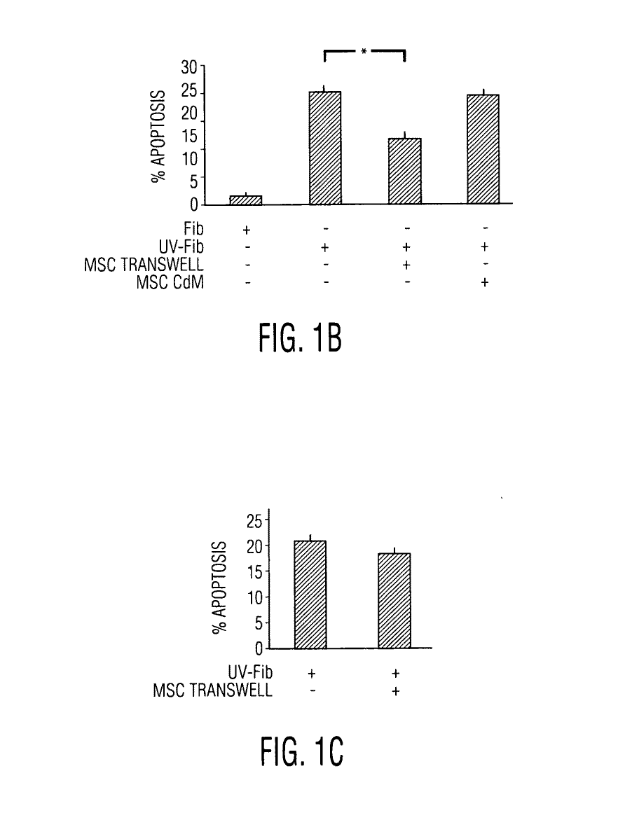

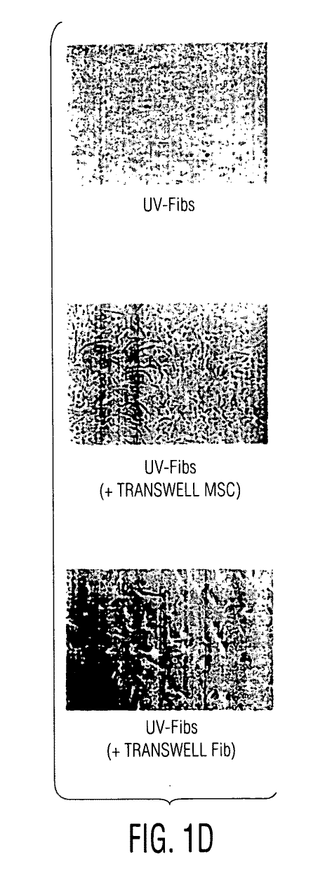

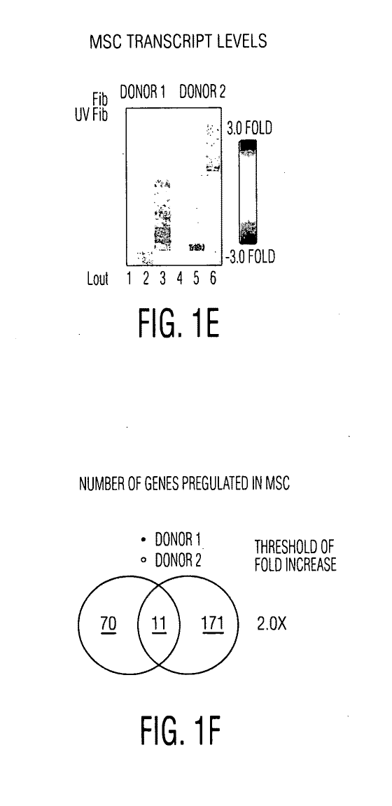

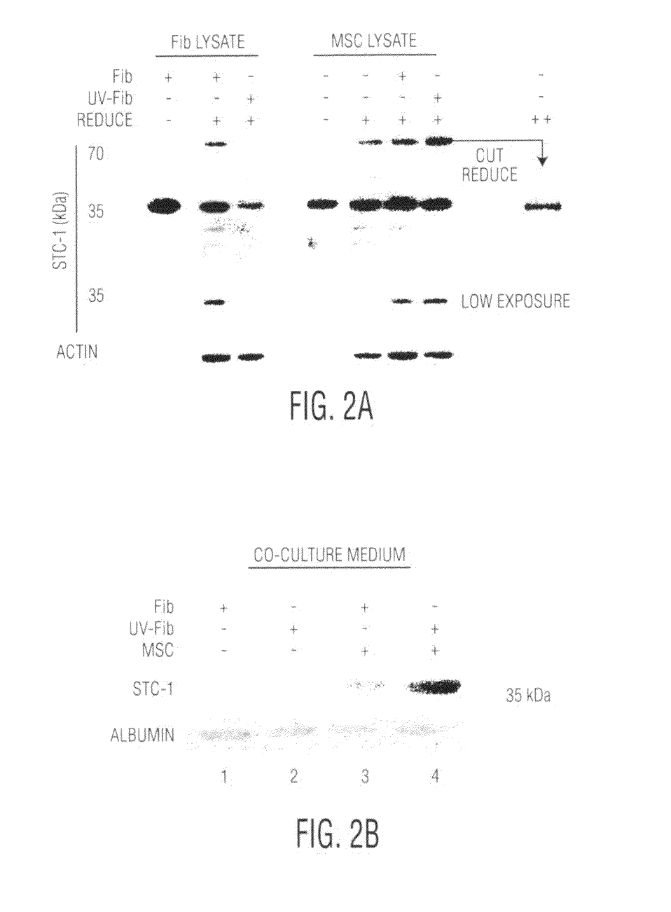

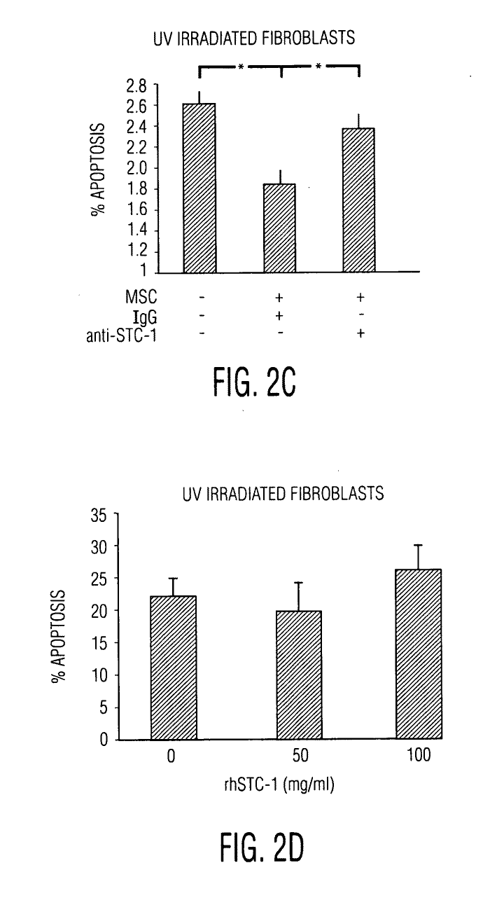

To investigate whether soluble factors derived from MSCs could reduce apoptosis, we first irradiated fibroblasts grown on a transwell filter with a sufficient dose of UV light (50 J/m²) to induce apoptosis in 15%-30% of the cells in 48 hours. We then placed the irradiated fibroblasts in coculture with MSCs incubated in a standard six-well dish, such that the two cell populations were 1 mm apart and separated by the 0.4 μm pores of the transwell membrane. Forty-eight hours following UV irradiation, a significant number of the fibroblasts had undergone apoptosis as assayed by annexin V and PI labeling (FIG. 1A, 1B [Schindl et. al., 1998, J Photochem Photobiol B 44:97-106; Wilkins et. al., 2002, Cytometry 48:14-1 9; Zhan et. al., 2002, Oncogene 21:5335-5345]. The use of annexin V-PI staining as a quantitative measure of apoptosis was validated by confirming other hallmarks of apoptosis, such as DNA degradation, TUNEL staining, nuclei defragmentation, and uptake of mitochondrial dye (supporting information FIG. 1). When the irradiated fibroblasts were cocultured with MSCs in the transwell, the level of apoptosis of the fibroblasts was reduced by about half. Similar results were obtained with MSCs from four additional donors (not shown) and A549 lung cancer epithelial cells (supporting information FIG. 2A). In contrast, conditioned medium from naïve MSCs (MSC CdM) did not reduce apoptosis of irradiated fibroblasts (FIG. 1A, 1B). As expected, controls of nonirradiated fibroblasts had no effect in the transwell system with irradiated fibroblasts (FIG. 1C). Phase contrast imaging of the cultures corroborated the observations. Cultures of irradiated fibroblasts incubated alone became sparse as cells detached from the filter and the cells lost their typical spindle-shaped morphology (FIG. 1D, left panel), whereas those cocultured in transwells with MSCs remained dense and the cells retained a spindle-shaped morphology (FIG. 1D, middle panel). Again, coculture in the transwells with nonirradiated fibroblasts had no effect on irradiated fibroblast morphology (FIG. 1D, right panel). The results indicated that the MSCs had to be activated by soluble factors produced by the irradiated fibroblasts to produce one or more soluble factors that reduced apoptosis in the fibroblasts.

### Coculture With Irradiated Fibroblasts Changed the Transcriptome of MSCs

As a preliminary screen for soluble antiapoptotic factors produced by the MSCs, we used microarrays to identify changes in the MSC transcriptome following incubation with UV-irradiated fibroblasts. FIG. 1D displays a heat map analysis of genes shared between two MSC donors after being filtered for genes that were upregulated or downregulated by more than twofold when cocultured with irradiated fibroblasts. MSCs from one donor upregulated 70 genes, whereas MSCs from a second donor upregulated 171 (FIG. 1F). The variation in data with the MSCs from the two donors probably reflects different degrees of apoptosis induced by the same exposure to irradiation and differences in the rates of proliferation of the MSCs under the coculture conditions, an example of the rapid changes in the transcriptome of MSCs as they are expanded in culture [Larson et al., 2008, Stem Cells 26:193-201]. Of special interest were the 11 upregulated genes common to both donors (FIG. 1F; Table 1). Of these 11 shared genes, only STC-1 encoded a secreted protein.

TABLE 1
Gene transcripts that are upregulated in multipotent stromal cells when
cocultured with irradiated fibroblasts*

| | | | Fold Increase | |
|---|---|---|---|---|
| Gene name | Secreted | Accession No. | Donor 1 | Donor 2 |
| Comichon homolog 3 | No | AF070524 | 4.17 | 4.62 |
| (Drosophila) | | | | |
| Endothelial cell-specific | No | NM_007036 | 2.2 | 3.99 |
| molecule 1 | | | | |
| Ets variant gene 1 | No | BE881590 | 2.52 | 4.02 |
| Hypothetical protein | No | BC006236 | 3.31 | 3.89 |
| MGC11324 | | | | |
| IGF-II mRNA-binding | No | AU160004 | 5.03 | 3.62 |
| protein 3 | | | | |
| | No | NM-006547 | 2.28 | 3.54 |
| Integrin, alpha 2 | No | NM_002203 | 3.57 | 2.28 |
| (CD49B, alpha 2 | | | | |
| subunit of VLA-2 | | | | |
| receptor) | | | | |
| | No | N95414 | 3.38 | 2.51 |
| Matrix | No | NM_002421 | 5.55 | 4.68 |
| metallopeptidase 1 | | | | |
| (interstitial collagenase) | | | | |
| Phosphatidylinostinol | No | BF308645 | 2.89 | 2.41 |
| 3.4.5-trisphosphate- | | | | |
| dependent RAC | | | | |
| exchanger 1 | | | | |
| | | AL445192 | 2.04 | 2.55 |
| Protein tyrosine | No | NM_015967 | 3.28 | 3.52 |
| phosphatase, | | | | |
| non-receptor | | | | |
| type 22 (lymphoid) | | | | |
| Ras-induced | No | BP062629 | 2.91 | 2.83 |
| senescence 1 | | | | |
| Stanniocalcin-1 | Yes | AB00520 | 3.49 | 4.6 |
| | | U46768 | 2.83 | 2.94 |
| | | NM_003155 | 3.04 | 5.55 |
| | | AW003173 | 2.78 | 6.96 |

*Threshold for upregulation, twofold.

STC-1 Expression and Secretion is Unregulated by MSCs in Cocultures with UV-Irradiated Fibroblasts

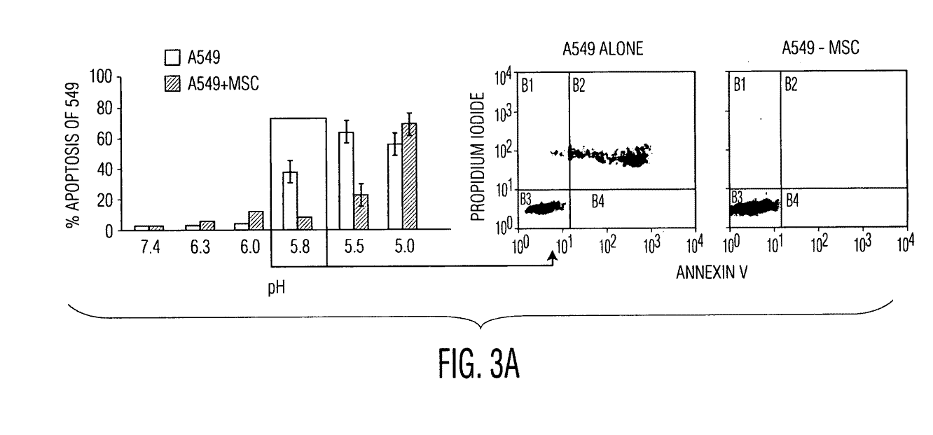

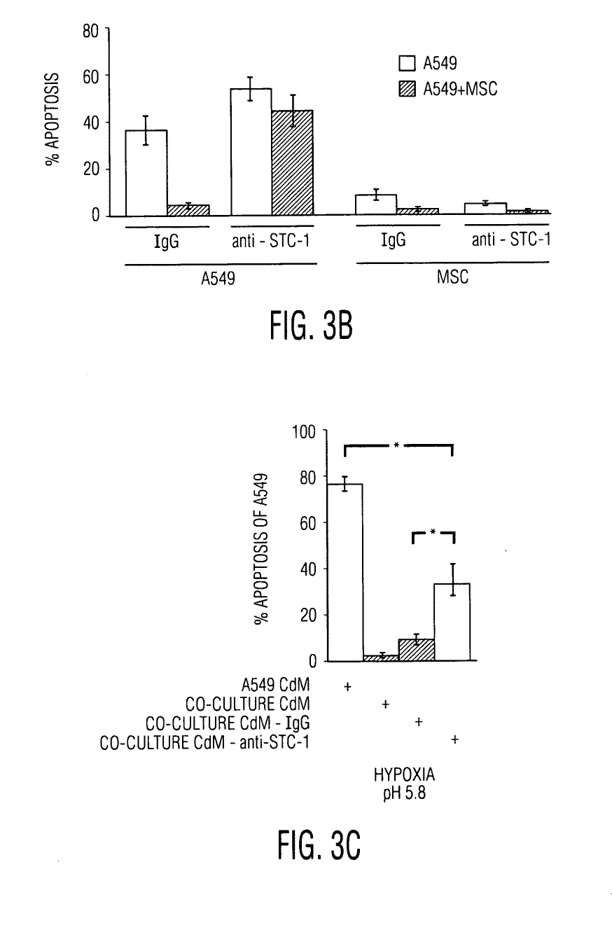

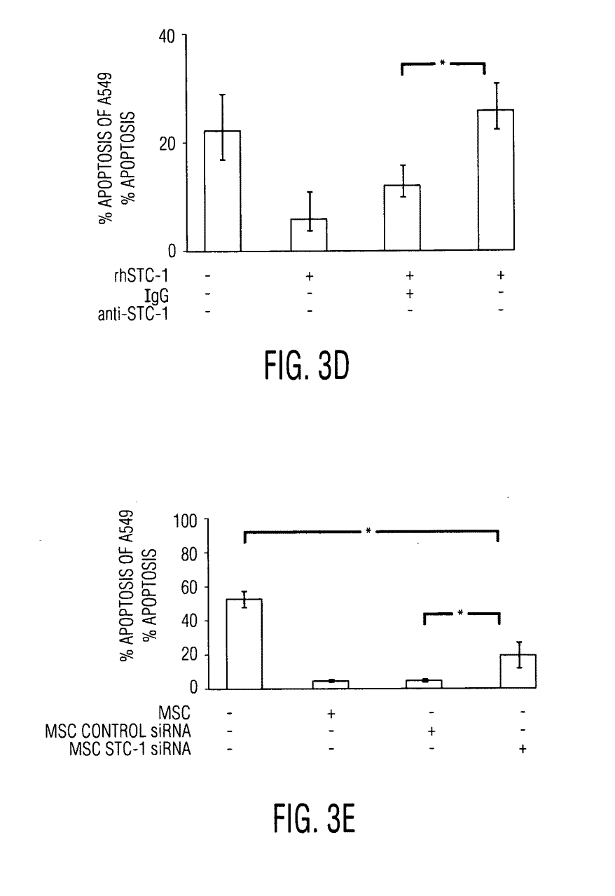

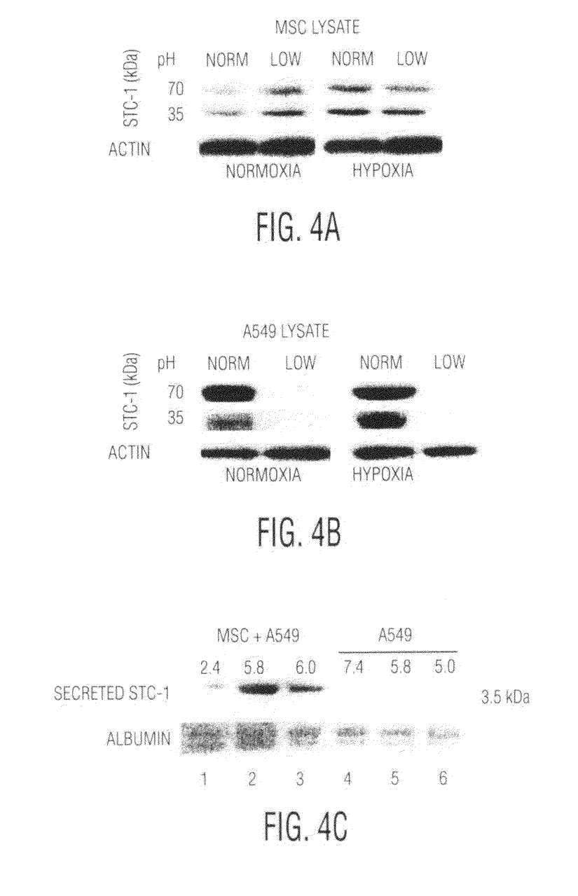

We explored next the possibility that secretion of STC-1 could account for the antiapoptotic effects of MSCs in the transwell system. Western blot assays with a polyclonal antibody for STC-1 demonstrated that cell lysates from naïve fibroblasts and MSCs contained a cross-reacting band of 35 kDa, the expected size of the protein [Varghese et. al., 2002, Endocrinology 143:868-876] (FIG. 2A, 2B). The lysates also contained a 70-kDa form of the protein that convened to 35 kDa after more extensive reduction (FIG. 2A). The specificity of the antibody for Western blots was demonstrated by preabsorbing the antibody with rhSTC-1 (supporting information FIG. 3A) and by confirming reactivity with both 35- and 70-kDa forms of rhSTC-1 (supporting information FIG. 3B). Also, Western blots with the antibody demonstrated decreased levels of the protein after the MSCs were transduced with an siRNA for STC-1 (supporting information FIG. 4). After irradiation, the STC-1 levels in lysates of fibroblasts decreased (FIG. 2A, lanes 2, 3). In contrast, there was an increase in the STC-1 content of MSCs after coculture with naïve fibroblasts (FIG. 2A, lane 6) and a greater increase after coculture with UV-irradiated fibroblasts, corroborating our microarray data (FIG. 2A, lane 7). (For clarity, a lower exposure of the 35-kDa band is shown in the lower panel.) Western blot assays of conditioned medium indicated that MSCs cocultured with UV-irradiated fibroblasts showed a marked increase in STC-1 secretion relative to MSCs cultured with nonirradiated fibroblasts or irradiated fibroblasts cultured alone (FIG. 2B). Addition of the antibody against STC-1 decreased the antiapoptotic effects of MSCs in cocultures with irradiated fibroblasts (FIG. 2C). An isotype control of IgG had no effect (FIG. 2C). Similar results were obtained with a second commercial source of antibody to STC-1 (not shown). Surprisingly, rhSTC-1 alone was unable to reduce apoptosis of the UV-irradiated fibroblasts (FIG. 2D). These results were confirmed by treating UV irradiated A549 cells with rhSTC-1, indicating that the effect was not cell type-specific (supporting information FIG. 2B). Therefore, the results suggested that in cocultures with irradiated fibroblasts, STC-1 was a necessary but not sufficient factor to explain the antiapoptotic effects of the MSCs.

MCSs Reduce Apoptosis in Cocultures with a Lung Epithelial Line (A549)

To extend and confirm the above observations we established a model of injury in which apoptosis was induced in lung epithelial cancer cells (A549). Preliminary experiments demonstrated that incubation of the A459 cells in 1% oxygen did not induce apoptosis. Similar results were previously observed with cultured cardiomyocytes [Graham et al., 2004, J Exp Biol. 207:3189-3200; Kim et. al., 2006, Am J Physiol Heart Circ Physiol 290:H2024-H2034]; therefore, we induced apoptosis in the A549 cells by incubation in hypoxia at low pH, conditions that were used to induce apoptosis in the cardiomyocytes and that often accompany reduced oxygen conditions in vivo as a result of increased lactate accumulation [Gladden, 2001, Proc Natl Acad Sci USA 98:395-397]. Cultures of A549 cells became apoptotic when incubated for 24 hours in 1% oxygen at pH 5.8 or 5.5 (FIG. 3A). MSCs cultured alone did not undergo apoptosis under the same conditions (FIG. 3B, right panel), but both A549 cells and MSCs underwent apoptosis when the pH was decreased further to 5.0 under hypoxic conditions. Coculture of A549 cells in transwells with MSCs reduced the apoptosis observed with hypoxia in medium adjusted to pH 5.8 or 5.5 but not if it was adjusted to pH 5.0 (FIG. 3A). Similar results were obtained with MSCs from two additional donors (not shown). Addition of antibodies to STC-1 reversed the antiapoptotic effects of the MSCs on the A549 cells under hypoxia and pH 5.8 (FIG. 3B, left panel). To confirm that the MSCs secreted an antiapoptotic factor or factors, cocultures of MSCs and A549 cells were incubated for 24 hours under hypoxia at pH 5.8. The conditioned medium (coculture CdM) was transferred to fresh cultures of A549 cells that were then incubated for 24 hours under hypoxia at pH 5.8. The coculture CdM inhibited apoptosis in the A549 cells. The antiapoptotic effects of the coculture CdM were blocked partially by antibodies to STC-1 but not by a control of IgG (FIG. 3C). Apoptosis of A549 cells incubated under hypoxia at pH 5.8 was also inhibited by rhSTC-1, and the effects were reversed by anti-STC-1 but not by control IgG (FIG. 3D). In further experiments, MSCs were transduced with siRNA for STC-1. The siRNA decreased synthesis of the protein by MSCs (supporting information FIG. 4). The MSCs expressing the siRNA were less effective than control MSCs in decreasing apoptosis of A549 cells in the transwell experiment, but the reversal was only partial (FIG. 3E).

### STC-1 Expression and Secretion in Cocultures of MSCs and A549 Cells

Western blot assays of cell lysates indicated that the levels of STC-1 in MSCs were increased by incubating the cells in 1% oxygen at pH 7.4 or 5.8 (FIG. 4A). In contrast, STC-1 was not detected in A549 cells after incubation at pH 5.8 under either normoxic or hypoxic conditions, even when the blot was overexposed (FIG. 4B). As expected, increased levels of secreted STC-1 were present in conditioned medium from cocultures of MSCs and A549 cells incubated under hypoxia at pH 5.8 (FIG. 4C), conditions under which the MSCs reduced the apoptosis of A549 cells. Secreted STC-1 levels were not affected by culturing MSCs with A549 cells under normal conditions (not shown).

### MCSs Restore Intracellular STC-1 in Injured Fibroblasts and A549 Cells After Rescue of Apoptosis

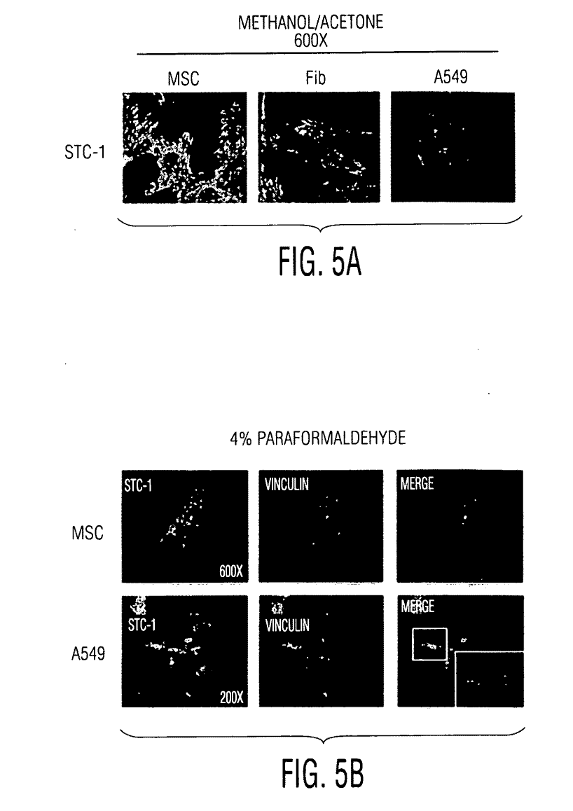

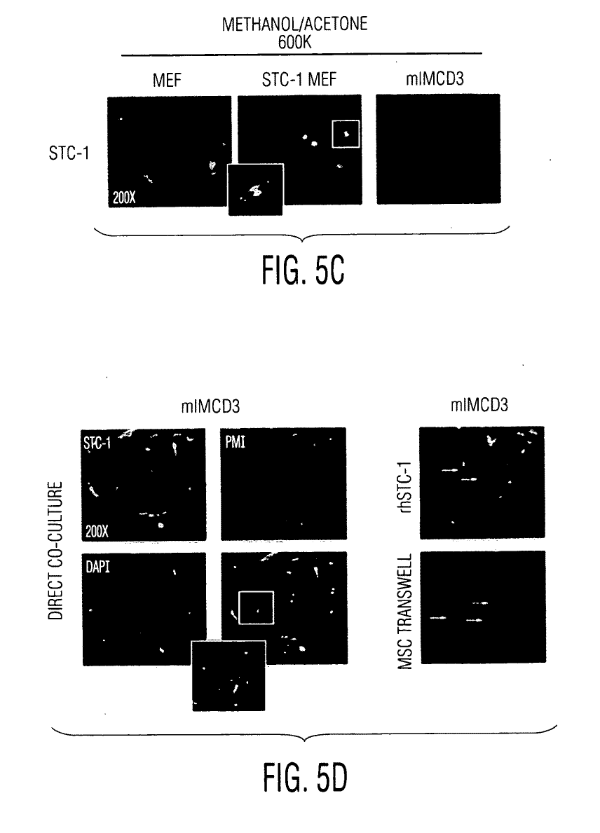

To determine the intracellular distribution of STC-1 in the cultures, the cells were examined by immunocytochemistry. STC-1 immunoreactivity was not detected in nonpermeabilized cells (supporting information FIG. 5A), indicating that STC-1 was not present in the extracellular matrix or the plasma side of the cell membrane. After the paraformaldehyde-fixed cells were permeabilized, or cells were fixed with methanol/acetone, STC-1 immunoreactivity was observed throughout the cytoplasm. STC-1 also appeared to be enriched in a pattern reminiscent of focal adhesions in all three cell types (FIG. 5A). The enrichment of STC-1 in focal adhesions was confirmed by colabeling the cells with antibodies to STC-1 and the actin-binding protein, vinculin (FIG. 5B). Preabsorption of the antibody with rhSTC-1 abolished immunoreactivity (supporting information FIG. 5B). We observed similar distribution of STC-1 in mouse embryonic fibroblasts (MEFs) obtained from wild-type or STC-1 overexpressing mice (STC-1-MEF) in that the cells also showed a focal adhesion-like peripheral staining pattern (FIG. 5C). Perinuclear accumulation of the STC-1 was also seen in the STC-1-MEFs, an observation consistent with synthesis of the protein in the rough endoplasmic reticulum (FIG. 5C, inset). Mouse inner medullary collecting duct cells, which are known to express very low amounts of STC-1 [Ellard et. al., 2007, Mol Cell Endocrinol 264:90-101; Sazonova et al., 2008, Am J Physiol 294:F7-F794], were negative for cytoplasmic and focal adhesion staining (FIG. 5C, right panel). To determine the fate of STC-1 secreted by MSCs, MSCs were cocultured with IMCD3 cells that do not express STC-1. In the cocultures, the MSCs were identified by presence of PML, a nuclear protein with a distinct punctate distribution [Block et. al., 2006, Mol Cell Biol 26:8814-8825]. The mouse IMCD3 cells were identified by the presence of pericentromeric heterochromatin and negative PML staining. In cocultures, the PML-negative IMCD3 cells acquired increased cytoplasmic immunoreactivity and clear focal adhesion enrichment of STC-1 (FIG. 5D, left panel). The same observation was made when IMCD3 cells were cocultured with MSCs in transwell culture or treated with rhSTC-1 (FIG. 5D, right panel). To corroborate these findings, A549 cells were transfected transiently with a construct expressing STC-1. The transfected cells showed more pronounced punctate foci (supporting information FIG. 5C).

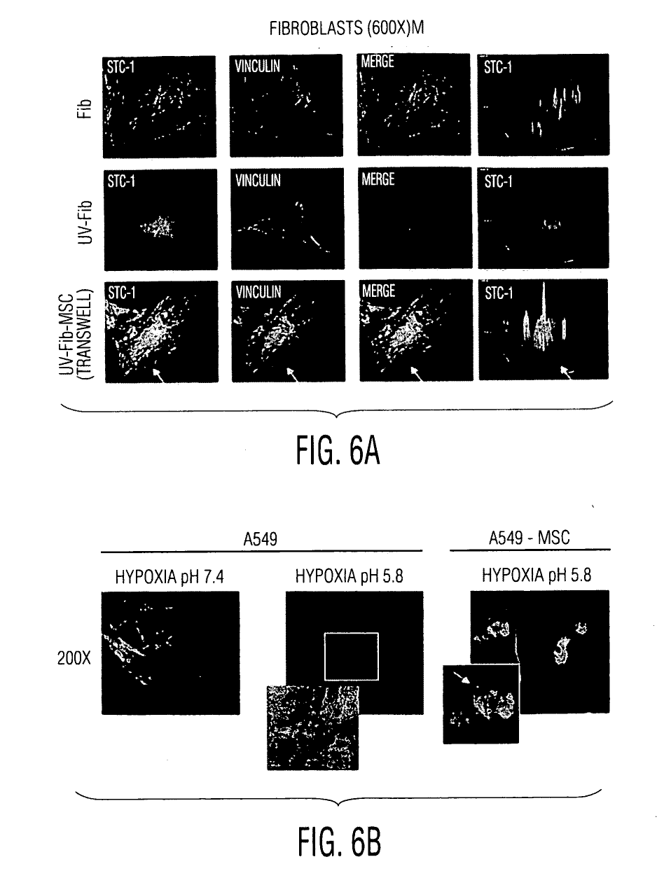

Irradiation of fibroblasts altered the distribution of STC-1. The protein was found near the nucleus or in vesicles (not shown), or it had disappeared altogether (FIG. 6A middle row), despite no change in the location of vinculin (FIG. 6A, middle row). After UV-irradiated fibroblasts were cocultured with MSCs, STC-1 was again colocalized with vinculin at focal adhesions of the fibroblasts (FIG. 6A, bottom row, arrows point to focal adhesions). Surface plot diagrams of STC-1 staining intensity are provided to confirm our observations. Similarly, incubation of A549 cells under hypoxia at low pH resulted in no detectable cytoplasmic STC-1; however, coculture with MSCs restored the intracellular distribution.

Coculture of MSCs with previously irradiated fibroblasts enabled us to assess both the antiapoptotic effects of MSCs and the effects of apoptotic cells on MSCs under the normal conditions for culture of MSCs. In effect, the system simulated conditions in vivo in which MSCs tend to home to injured cells and tissues, including those injured by irradiation [Mouiseddine et. al., 2007, Br J Radiol 80 (spec. no. 1):S49-S55. The results demonstrated that exposure of MSCs to the irradiated fibroblasts changed their patterns of expressed genes. Although substantial donor variation was observed, transcripts for 11 common genes were upregulated twofold or more in both donor cell populations. Because the signals exchanged by the MSCs and fibroblasts were transmitted through a 0.4-μm pore transwell filter, the microarray data from the MSCs were queried for upregulation of transcripts for secreted proteins. The most abundant transcripts for a secreted protein that was upregulated by twofold or more were transcripts from the gene encoding STC-1. The use of microarrays to identify secreted molecules had limitations, and thus, the data did not reflect all of the important changes in the MSC secretome. For example, the microarray data would not have reflected changes in low-abundance transcripts and can underestimate the changes in some transcripts in MSCs [Larson et. al., 2008, Stem Cells 26:193-201]. Furthermore, the microarrays could not identify changes that occurred at the post-transcriptional level; however, they provided a useful indication of one candidate to examine further, STC-1.

Exposure of MSCs to irradiated fibroblasts increased both synthesis and secretion of STC-1. Interestingly, the upregulation of STC-1 by MSCs was not as apparent in cell lysates as in conditioned medium, indicating that the rate of the secretion of the protein may be increased as well. Antibodies targeted against STC-1 decreased the antiapoptotic effect of the MSCs. Treatment of irradiated fibroblasts with excess rhSTC-1 was unable to reduce apoptosis when rhSTC-1 was used at the same concentrations that gave positive results for A549 cells grown in ischemic conditions. Furthermore, rhSTC-1 had no effect on irradiated A549s, indicating that the effect was not cell type-specific. Thus, STC-1 was required but not sufficient to reduce apoptosis and may be enhancing or antagonizing the effects of other secreted factors from the MSC CdM. For example, pretreatment of endothelial cells with STC-1 impaired hepatocyte growth factor-induced phosphorylation of focal adhesion kinase [Ziot et. al., 2003, J Biol Chem 278:47654-47659]. Also, pretreatment of a macrophage-like cell decreased intracellular calcium accumulation in response to two cytokines, monocyte chemotactic protein and stromal cell-derived factor-1 [Kanellis et. al., 2004, Am J Physiol 286:F356-F362]. Previous studies showed that STC-1 was upregulated in cancer cells during hypoxia [Westberg et. al., 2007, Stroke 38:1025-103; Westberg et. al., 2007, Am J Physiol Heart Circ Physiol 293:H1766-H1771; Yeung et. al., 2005, Endocrinology 146:4951-4910]. Our results also demonstrated that MSCs decreased apoptosis in cocultures with a line of pulmonary epithelial cells in which apoptosis was produced by hypoxia under acidic conditions. We then asked whether upregulation of STC-1 by MSCs was responsible for the effect. Interestingly, when MSCs were cocultured with A549 cells under hypoxia and acidosis, expression of STC-1 was upregulated in MSCs but abolished in A549 cells. Therefore, the upregulation of STC-1 in MSCs was independent of crosstalk with the A549 cells. Antibody blocking and siRNA knockdown of STC-1 within MSCs partially impaired the ability of the MSCs to reduce apoptosis. The partial reversal may be the result of inefficient knockdown or blocking of STC-1 or may indicate the involvement of other MSC-derived factors in the reduction of apoptosis. In this system, STC-1 was both necessary and sufficient to reduce apoptosis of the ischemic lung cells. The results were consistent with previous reports that STC-1 was upregulated by hypoxia in cancer cells.

Previous studies, observed the presence of STC-1 in many different cellular compartments, frequently as a receptor/ligand complex. In sections of mouse kidney, STC-1 was present throughout the cytoplasm with apparent enrichment within mitochondria [McCudden et. al., 2002, J Biol Chem 277:45249-45258] In cardiomyocytes overexpressing a STC-1-FLAG fusion protein, STC-1 was seen in mitochondria [Westberg et. al., 2007, Am J Physiol Heart Circ Physiol 293:H1766-H1771]. STC-1 was also found in the nucleus in cardiomyocytes [Sheikh-Hamad et. al., 2003, Am J Physiol Heart Circ Physiol 285:H442-H448] and in mouse lactiferous duct cells during pregnancy [Hasilo et. al., 2005, Am J Physiol 289:E634-E642]. Here we observed pancytoplasmic staining of STC-1 with enriched immunoreactivity at focal adhesion plaques. Focal adhesion distribution was observed in all three human cell types, as well as two primary MEF cultures. The presence of STC-1 in focal adhesion plaques was supported by colocalization with the actin-binding protein vinculin. Following injury of both cell types in each condition, STC-1 was depleted from focal adhesions, but localization was restored after rescue by MSCs. We provided evidence that secreted STC-1 localized to focal adhesions of STC-1-null IMCD3 target cells, indicating that STC-1 leaves the cell prior to localizing to focal adhesions. Therefore, MSC-derived STC-1 may localize to focal adhesions in injured cells and promote viability by a mechanism that has yet to be determined.

The importance of STC-1 in mammalian systems is not well understood; however, a growing body of evidence indicates that it may be a critical stress response protein. STC-1 upregulation has been observed in multiple models of injury, including the ischemic brain [Zhang et al., 2000, Proc Natl Acad Sci USA 97:3637-3642] obstructed kidney [Kanellis et. al., 2004, Am J Physiol 286:F356-F362], and the hypoxia-preconditioned heart and brain [Westberg et al., 2007, Am J Physiol Heart Circ Physiol 293:H1766-H1771]. Furthermore, the STC-1 gene is activated readily by multiple cytokines [Zlot et. al., 2003, J Biol Chem 278:47654-47659; Kanellis et. al., 2004, Am J Physiol 286:F356-F362; Holmes et. al., 2008, Cell Signal 20:569-579). Thus, the upregulation of STC-1 in MSCs by irradiated fibroblasts is likely due to the presence of similar cytokines released by the injured cells. These factors have yet to be determined. Previous observations on pro- or antiapoptotic effects of STC-1, however, were inconsistent. For example, increased expression after hypoxia-preconditioning of heart and brain suggested that STC-1 was antiapoptotic [Westberg et. al., 2007, Stroke 38:1025-103]. In contrast, STC-1 was proapoptotic in chondrocytes during bone development [Wu et. al., 2006, J Biol Chem 281:5120-5127], and transgenic mice overexpressing STC-1 had defects in bone growth [Varghese et al., 2002, Endocrinology 143:868-876; Filvaroff et al., 2002, Endocrinology 143:3681-3690]. We have demonstrated that MSC-derived STC-1 can have antiapoptotic effects. Thus, the cytoprotective effects of MSCs may be explained in part by the upregulation of STC-1.

### Example 2

### Materials and Methods

Androgen-sensitive human prostate cancer cell line LNCaP clone FGC, androgen independent prostate cancer cell line PC3, and progesterone responsive human breast cancer cells T47D were purchased from American Type Culture Collection (Rockville, Md.). Cells were maintained in RPMI 1640 medium supplemented with heat inactivated 10% FBS, 2 mM glutamine and 50 μg/mL gentamicin. The effects of STC-1 on the steroid induced growth were tested as follows:

Cells were plated 2,000 cells/well in 100 μl. growth medium in poly-D-lysine coated 96 well plates. After incubation for 2 days, cells were steroid starved for 48 hrs in steroid reduced medium (phenol red-free RPMI 1640 medium containing 5% charcoal/dextran treated FBS, 2 mM glutamine and 50 μg/mL gentamicin). Cells were then exposed to 10 nM of 5α-dihydrotetosterone (DHT) for LNCaP or PC3 cells or 100 nM progesterone for T47D cells for an additional 48 hrs. During steroid exposure recombinant STC-1 (Biovendor, Candler, N.C.) or vehicle control were added to the media. Final cell numbers were quantified by measuring absorbance as determined by MTT assay (Vybrant® MTT Cell Proliferation Assay Kit, Invitrogen) or Cyquant assay (CyQUANT® Cell Proliferation Assay Kit, Invitrogen).

hMSCs from bone marrow were obtained from the Center for the Preparation and Distribution of Adult Stem Cells (formerly http://www.som.tulane.edu/genetherapy/distribute. shtml; currently msc@medicine.tamhsc.edu). hMSCs were cultured in complete culture medium (CCM) that consisted of α-minimal essential medium (α-MEM; Invitrogen, Carlsbad, Calif.), 17% fetal bovine serum (FBS; lot-selected for rapid growth of MSCs; Atlanta Biologicals, Inc; Norcross, Ga.), 100 units/mL penicillin, 100 μg/mL streptomycin, and 2 mM L-glutamine (Invitrogen). Cells were plated 2,000 cells/well in 100 μL CCM in 96 well plates. After incubation for 2 days, cells were steroid starved for 48 hrs in steroid reduced medium (phenol red-free RPMI 1640 medium containing 5% charcoal/dextran treated FBS, 2 mM glutamine and 50 μg/mL gentamicin). Cells then were exposed to 10 nM of 5α-dihydrotetosterone (DHT) and STC-1 or vehicle control for an additional 48 hrs. Final cell numbers were quantified by MTT assay (Vybrant® MTT Cell Proliferation Assay Kit, Invitrogen).

### Results and Discussion

Prostate cancer therapies aim either to eliminate the cancer itself or to prevent cancer cell proliferation. If the cancer is localized in the prostate, the most common therapies are radiation and/or prostatectomy. If the cancer grows beyond the edge of the prostate, radiation and/or surgery are no longer appropriate. At this stage, the standard treatment for prostate cancer is androgen deprivation therapy. Androgens, such as testosterone, are essential for prostate tumor growth. Hence, hormonal therapies employ androgen-deprivation essentially to starve cancer cells.

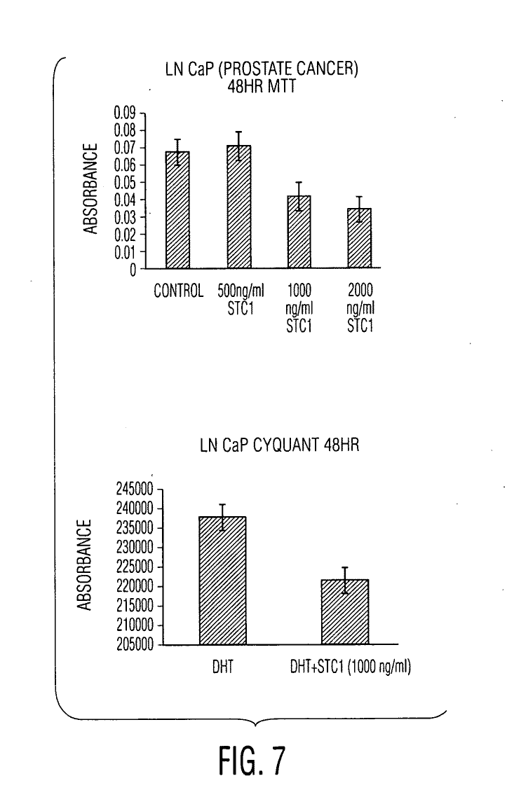

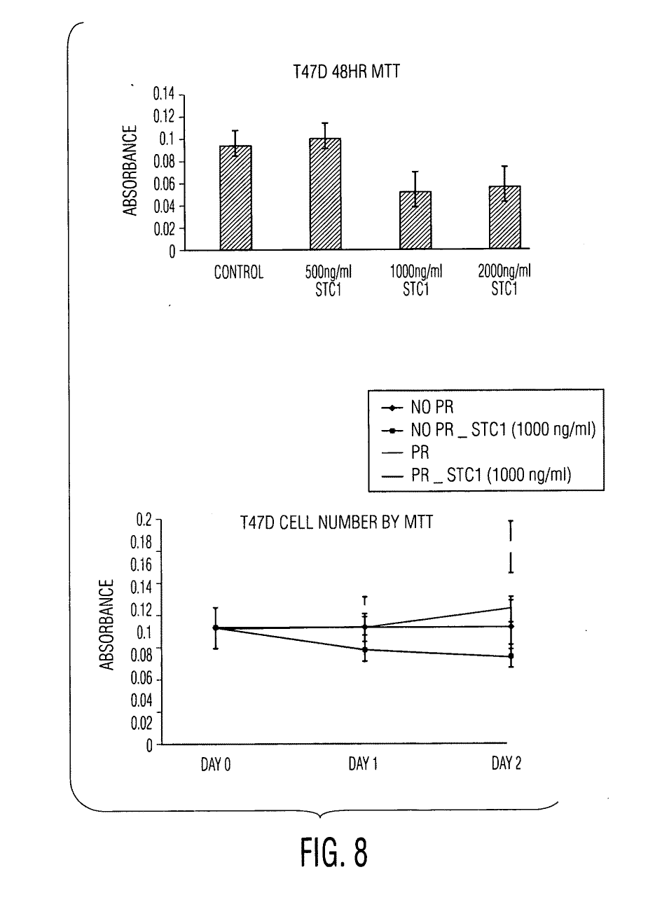

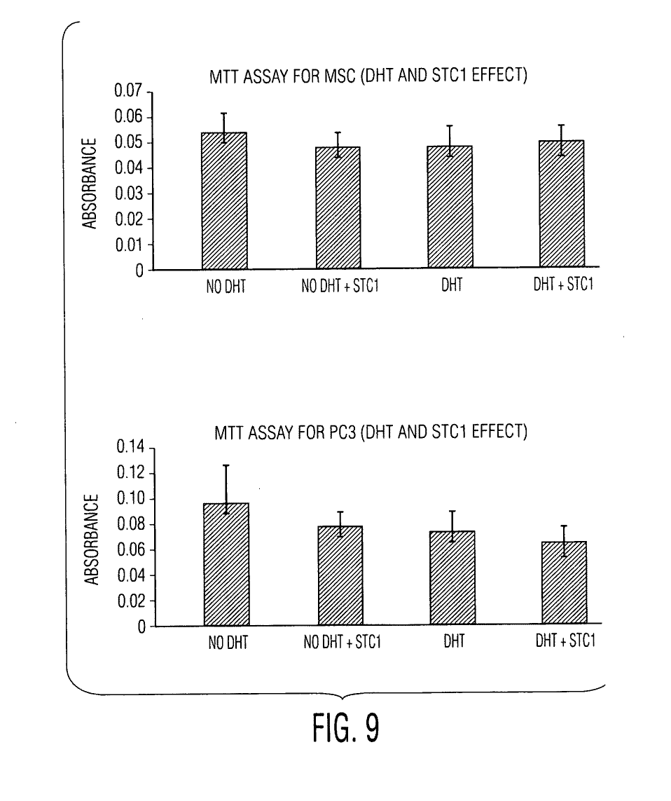

Growth stimulation of prostate cancer cells by androgens requires p66Shc mediated mitochondrial ROS production. STC-1 repressed 5α-dihydrotetosterone (DHT; biologically active metabolite of the hormone testosterone) dependent proliferation of prostate cancer cell line LNCaP (FIG. 7). STC-1 also reduced another hormone dependent cancer proliferation. The STC-1 treatment reduced the progesterone dependent breast cancer cell (T47D) proliferation (FIG. 8). STC-1 treatment did not affect steroid independent proliferation of PC3 prostate cancer cell line or growth of hMSCs (FIG. 9). This indicated that the effect of STC-1 in cell proliferation specifically limited to the hormone dependent cancer cell proliferations. The results demonstrated noble prostate cancer treatment using recombinant STC-1 protein. STC-1 may deprive the androgen stimulation to the cancer cells by modulating the ROS dependent downstream signaling of steroid receptors. The application may be expanded to other cancer treatments including hormone dependent breast cancers.

The disclosures of each and every patent, patent application, and publication cited herein are hereby incorporated herein by reference in their entirety to the same extent as if each patent, patent application, and publication were incorporated individually by reference.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations. Thus, the invention may be practiced other than as particularly described and still be within the scope of the accompanying claims.

What is claimed is:

1. Mesenchymal stem cells which have been cultured under conditions to express an increased amount of at least one anti-apoptotic protein compared to the amount of said at least one anti-apoptotic protein expressed by an otherwise identical population of mesenchymal stem cells which have not been cultured under conditions to express an increased amount of said at least one anti-apoptotic protein.

2. The cells of claim 1 wherein said at least one anti-apoptotic protein is STC-1.

3. The cells of claim 1 wherein said at least one anti-apoptotic protein is STC-2.

4. The cells of claim 1 wherein said mesenchymal stem cells were contacted with apoptotic cells.

5. A method of treating a disease, disorder, or condition associated with apoptosis in a mammal, comprising:
administering to said mammal mesenchymal stem cells which have been cultured under conditions to express increased amounts of at least one anti-apoptotic protein compared to the amount of said at least one anti-apoptotic protein expressed by an otherwise identical population of mesenchymal stem cells which have not been cultured under conditions to express an increased amount of said at least one anti-apoptotic protein, wherein said mesenchymal stem cells are administered in an amount effective to treat said disease, disorder, or condition associated with apoptosis in said mammal.

6. The method of claim 5 wherein said at least one anti-apoptotic protein is STC-1.

7. The method of claim 5 wherein said at least one anti-apoptotic protein is STC-2.

8. A method of treating a cancer patient undergoing anti-cancer treatment in order to prevent, limit, or treat damage to non-cancerous cells in said patient, comprising:
administering to said patient mesenchymal stem cells which have been cultured under conditions to express increased amounts of at least one anti-apoptotic protein compared to the amount of said at least one anti-apoptotic protein expressed by an otherwise identical population of mesenchymal stem cells which have not been cultured under conditions to express an increased amount of said at least one anti-apoptotic protein, wherein said mesenchymal stem cells are administered in an amount effective to prevent, limit, or treat damage to non-cancerous cells in said patient.

9. The method of claim 8 wherein said at least one anti-apoptotic protein is STC-1.

10. The method of claim 8 wherein said at least one anti-apoptotic protein is STC-2.

11. The method of claim 8 wherein said anti-cancer treatment comprises administering to said patient tumor necrosis factor-related apoptosis-inducing ligand (TRAIL).

12. The method of claim 8 wherein said anti-cancer treatment comprises administering to said patient mesenchymal stem cells which express tumor necrosis factor-related apoptosis-inducing ligand.

13. A method of treating a cancer in which disruption of reactive oxygen species results in anti-cancer activity, in a patient, comprising:
administering to said patient at least one anti-apoptotic protein, wherein said at least one anti-apoptotic protein is administered in an amount effective to treat said cancer in which disruption of reactive oxygen species results in anti-cancer activity.

14. The method of claim 13 wherein said at least one anti-apoptotic protein is STC-1.

15. The method of claim 13 wherein said cancer in which disruption of reactive oxygen species results in anti-cancer activity is prostate cancer.

16. The method of claim 13 wherein said cancer in which disruption of reactive oxygen species results in anti-cancer activity is breast cancer.

17. A method of treating a cancer in which disruption of reactive oxygen species results in anti-cancer activity, in a patient comprising:
administering to said patient mesenchymal stem cells which have been cultured under conditions to express increased amounts of at least one anti-apoptotic protein compared to the amount of said at least one anti-apoptotic protein expressed by an otherwise identical population of mesenchymal stem cells which have not been cultured under conditions to express an increased amount of said at least one anti-apoptotic protein, wherein said mesenchymal stem cells are administered in an amount effective to treat said cancer in which disruption of reactive oxygen species results in anti-cancer activity.

18. The method of claim 17 wherein said cancer in which disruption of reactive oxygen species results in anti-cancer activity is prostate cancer.

19. The method of claim 17 wherein said cancer in which disruption of reactive oxygen species results in anti-cancer activity is breast cancer

20. The method of claim 17 wherein said at least one anti-apoptotic protein is STC-1.

21. A method of treating injury to a tissue resulting from lack of oxygen in a patient, comprising:
administering to said patient mesenchymal stem cells which have been cultured under conditions to express increased amounts of at least one anti-apoptotic protein compared to the amount of said at least one anti-apoptotic protein expressed by an otherwise identical population of mesenchymal stem cells which have not been cultured under conditions to express an increased amount of said at least one anti-apoptotic protein, wherein said mesenchymal stem cells are administered in an amount effective to treat said injury to a tissue resulting from lack of oxygen.

22. The method of claim 21 wherein said at least one anti-apoptotic protein is STC-1.

23. The method of claim 21 wherein said at least one anti-apoptotic protein is STC-2.

24. A method of treating or preventing organ death and/or loss of organ function in a patient comprising:
administering to said patient mesenchymal stem cells which have been cultured under conditions to express increased amounts of at least one anti-apoptotic protein compared to the amount of said at least one anti-apoptotic protein expressed by an otherwise identical population of mesenchymal stem cells which have not been cultured under conditions to express an increased amount of said at least one anti-apoptotic protein; wherein said mesenchymal stem cells are administered in an amount effective to treat or prevent organ death and/or loss of organ function in said patient.

25. The method of claim 24 wherein said organ death and/or loss of organ function results from loss of normal blood flow to said organ.

26. The method of claim 25 wherein said organ is the heart.

27. The method of claim 24 wherein said at least one anti-apoptotic protein is STC-1.

28. The method of claim 24 wherein said at least one anti-apoptotic protein is STC-2.

29. A method of treating or preventing cell apoptosis and/or cell death resulting from the introduction of one or more toxins into a patient, comprising:
administering to said patient mesenchymal stem cells which have been cultured under conditions to express increased amounts of at least one anti-apoptotic protein compared to the amount of said at least one anti-apoptotic protein expressed by an otherwise identical population of mesenchymal stem cells which have not been cultured under conditions to express an increased amount of said at least one anti-apoptotic protein, wherein said mesenchymal stem cells are administered in an amount effective to treat or prevent cell apoptosis and/or cell death resulting from the introduction of one or more toxins into said patient.

30. The method of claim 29 wherein said at least one anti-apoptotic protein is STC-1.

31. The method of claim 29 wherein said at least one anti-apoptotic protein is STC-2.

32. A method of treating unresolved inflammation in a patient, comprising:
administering to said patient mesenchymal stem cells which have been cultured under conditions to express increased amounts of at least one anti-apoptotic protein expressed by an otherwise identical population of mesenchymal stem cells which have not been cultured under conditions to express an increased amount of said at least one anti-apoptotic protein, wherein said mesenchymal stem cells are administered in an amount effective to treat said unresolved inflammation in said patient.

33. A method of treating a disease or disorder that is associated with excessive production of reactive oxygen species in a patient, comprising:
administering to said patient at least one anti-apoptotic protein in an amount effective to treat said disease or disorder that is associated with excessive production of reactive oxygen species in said patient.

34. The method of claim 33 wherein said at least one anti-apoptotic protein is STC-1.
